(19) United States
(12) Patent Application Publication
(10) Pub. No.: US 2026/0233020 A1
(43) Pub. Date: Aug. 13, 2026

(54) PHOTOBIOMODULATION DEVICE FOR ACCELERATING SURGICAL REHABILITATION AND METHOD THEREOF

(71) Applicant: ELIXIR MD INC., Irvine, CA (US)

(72) Inventor: Ewan Yassen, Irvine, CA (US)

(21) Appl. No.: 19/262,176

(22) Filed: Jul. 8, 2025

Related U.S. Application Data
(60) Provisional application No. 63/758,020, filed on Feb. 13, 2025.

(51) Int. Cl. A61N 5/06; G16H 10/60; G16H 20/40

(52) U.S. Cl. CPC A61N 5/06; G16H 10/60; G16H 20/40; A61N 2005/0626; A61N 2005/0652; A61N 2005/0659; A61N 2005/0663

(57) ABSTRACT

A photobiomodulation device for accelerating surgical rehabilitation and method thereof are disclosed. The device includes a mainframe mounted on a mobile structure, an irradiator with a plurality of treatment panels comprising a plurality of LEDs, and an adjustable expandable arm for positioning over a target anatomical region. The LEDs emit at least one of: red-light, blue light, yellow light, and infrared light at predefined wavelengths. The photobiomodulation device integrates one or more sensors for real-time measurement of temperature and distance, a user interface for selecting treatment protocols for treatment control, safety interruption, data logging, and LED calibration. A treatment control subsystem dynamically adjusts parameters based on temperature data and distance data and regulates emission to stimulate cellular functions such as mitochondrial activity, inflammation modulation, and tissue regeneration. The photobiomodulation device enhances therapeutic precision through intelligent feedback and calibration, enabling safer, personalized, and effective photobiomodulation therapy for postoperative recovery.

### CROSS REFERENCE TO RELATED APPLICATION(S)

This application claims the priority to and incorporates by reference the entire disclosure of U.S. provisional patent application bearing No. 63/758,020 filed on Feb. 13, 2025.

### TECHNICAL FIELD

Embodiments of the present disclosure relate to phototherapy and more particularly relate to a photobiomodulation device for accelerating surgical rehabilitation and a method thereof.

### BACKGROUND

In a realm of modern medicine and surgical procedures, achieving optimal pre-operative recovery and post-operative recovery is a critical factor in improving patient outcomes and minimizing complications. Traditional methods for enhancing recovery rely on pharmacological interventions, physical therapy, and passive healing, which is time-consuming and poses risks of side effects. Photobiomodulation therapy, also referred as low-level light therapy (LLLT), has emerged as a promising non-invasive technique that utilizes specific wavelengths of light to stimulate cellular activity, promote tissue regeneration, and reduce inflammation. However, current Photobiomodulation devices lack tailored features to address the specific demands of plastic surgery and general surgery.

The prior art in Photobiomodulation technology includes a range of handheld and fixed-light devices configured for general therapeutic applications. The handheld and fixed-light devices operate on limited wavelength ranges and provide fixed irradiance levels that may not be suitable for all tissue types and stages of recovery. Moreover, the handheld and fixed-light devices are bulky, cumbersome to use, and lack intelligent control systems to adjust treatment parameters dynamically. These shortcomings restrict efficacy, especially in delicate surgical contexts where precise energy delivery and controlled treatment are paramount.

Another drawback of existing Photobiomodulation devices is the inability to integrate seamlessly into surgical workflows. Many Photobiomodulation devices require significant operator intervention to configure treatment settings and are unable to provide targeted therapy for specific anatomical regions. Additionally, the Photobiomodulation devices lack robust safety features to prevent overheating and overexposure, increasing the risk of tissue damage and diminishing patient confidence. Such limitations have created a demand for a more advanced solution tailored specifically to the pre-operative needs and post-operative needs in the plastic surgery and general surgery.

Therefore, there is a need for a device that provides targeted and adaptive photobiomodulation therapy tailored specifically for pre-operative and post-operative recovery in the plastic surgery and general surgery. Such a device should overcome the limitations of prior art by providing multi-wavelength functionality, precise irradiance control, and a user-friendly interface.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a photobiomodulation device for accelerating surgical rehabilitation is disclosed.

In an embodiment, the photobiomodulation device comprises a mainframe, an irradiator, one or more sensors, a user interface, one or more microcontrollers, and a memory unit. In one aspect, the mainframe is operatively mounted on a mobile structure with a plurality of lockable wheels. The mainframe is configured to house at least one of: one or more electronic components and power sockets.

In other aspects, the irradiator is configured with a plurality of treatment panels operatively connected to the mainframe on a first side through an adjustable expandable arm. Each treatment panel of the plurality of treatment panels comprises a plurality of light-emitting diodes (LEDs). The plurality of LEDs comprises 280 surface-mounted LEDs arranged in a predefined matrix pattern. The plurality of treatment panels comprises five treatment panels, each treatment panel of the plurality of treatment panels capable of being folded inward to conform to a curvature of one of: a human face and body contour. The plurality of LEDs configured to emit at least one of: red-light, blue light, yellow light, and infrared light. Each LED of the plurality of LEDs operate at a predefined wavelength.

Yet other aspects, the predefined wavelengths of the red-light range between 623 nanometers (nm) and 643 nm. The predefined wavelengths of the blue light range between 407 nm and 427 nm. The predefined wavelengths of the yellow light range between 580 nm and 600 nm. The predefined wavelengths of the infrared light range between 825 nm and 845 nm. At least one of: the red-light, the blue light, the yellow light, and the infrared light is emitted in a pulsed mode with defined frequencies of 2 Hertz (Hz), 5 Hz, and 10 Hz at flashing interval times of 0.5 seconds(s), 0.2 s and 0.1 s, respectively. At least one of: the red-light, the blue light, the yellow light, and the infrared light is emitted in one of: a concurrent mode and a sequential mode, with multiple wavelength emission.

In another aspect, the adjustable expandable arm is configured to provide at least one of: vertical direction and horizontal direction to the irradiator for reaching a target anatomical region. The adjustable expandable arm is configured to rotate positive 80 degrees and negative 80 degrees from a longitudinal axis of the mainframe. The adjustable expandable arm is configured to tilt positive 15 degrees and negative 15 degrees from a pivotal point of the adjustable expandable arm.

Yet another aspect, the one or more sensors are operatively positioned on one or more treatment panels of the plurality of treatment panels. The one or more sensors are configured to determine at least one of: target anatomical region temperature during operation and distance between the irradiator and the target anatomical region for generating temperature data and distance data. The one or more sensors comprises at least one of: a temperature sensor embedded at a second side of the central treatment panel of the plurality of treatment panels to determine the target anatomical region temperature, and a distance sensor integrated at a third side of the central treatment panel of the plurality of treatment panels to determine the distance between the irradiator and the target anatomical region.

In other aspects, the user interface is operatively mounted on the mainframe at a first end, configured to control one or more treatment parameters. The one or more treatment parameters comprise at least one of: preset and named treatment designed protocols selection, wavelength selection, irradiance, energy delivered in per unit area, emission mode, irradiator configuration mode, one of: a flat mode and a curved mode of the irradiator, and one of: a sequential mode and a concurrent mode, at a time of selecting multiple wavelengths, and treatment time. The user interface is configured to provide at least one of: one or more clickable elements, one or more voice comment inputs, one or more touchscreen inputs, one or more gesture-based controls, one or more remote-control inputs, and one or more programmable physical buttons, comprise at least one of: a) a wavelength selector for choosing one or more wavelengths to be emitted by the irradiator, b) a power level selector for regulating the irradiance delivered to the target anatomical region, c) a treatment duration timer for setting the treatment time, d) a treatment dosage input for setting a dose of the energy delivered in per unit area, e) a mode selector for switching between pulsed mode and continuous mode, f) a treatment controller for one of: initiating, terminating, and pausing treatment, g) an irradiator mode selector for switching between one of: the flat mode and a curved mode of the irradiator, h) a treatment mode selector for switching between one of: the sequential mode and the concurrent mode, at the time of selecting multiple wavelengths, and treatment time, and i) a preset protocol selector for applying predefined treatment parameters based on one of: a treatment type and a body region.

Yet other aspects, the memory unit is operatively connected to the one or more microcontrollers, the memory unit comprises a set of computer-readable instructions in form of a plurality of subsystems. The memory unit is configured to be executed by the one or more microcontrollers. The plurality of subsystems comprises a data obtaining subsystem, a threshold-based safety interruption subsystem, a calibration subsystem, a data logging subsystem, and a treatment control subsystem.

In another aspect, the data obtaining subsystem is configured to obtain at least one of: the temperature data and the distance data from the one or more sensors in real time.

Yet another aspect, the threshold-based safety interruption subsystem is configured to trigger at least one of: one or more alerts and one or more termination commands of the irradiator if at least one of: the temperature data and the distance data falls outside a predefined threshold temperature data and a predefined operational distance range, respectively.

In other aspects, the calibration subsystem is configured to calibrate each LED of the plurality of LEDs by performing a comparative analysis between a real-time predefined wavelength and a factory-calibrated reference wavelength for detecting output deviations in the predefined wavelength. The calibration subsystem is configured to trigger one or more calibration alerts after every 500 hours of cumulative operation. The calibration subsystem is configured to perform one of: an automatic calibration and a manual calibration of the plurality of LEDs based on one of: predefined usage time and user initiation.

Yet other aspects, the data logging subsystem is configured to log rehabilitation session data, including at least one of: a timestamp, selected wavelengths, energy delivered per unit area, irradiance, emission mode, and sensor readings into one or more databases.

In another aspect, the treatment control subsystem is configured to regulate the one or more treatment parameters based on at least one of: the temperature data and the distance data for emitting at least one of: the red-light, the blue light, the yellow light, and the infrared light at the predefined wavelengths. The treatment control subsystem is configured to dynamically regulate treatment duration based on the real-time irradiance values and the selected energy delivered per unit area. The emitted at least one of: the red-light, the blue light, the yellow light, and the infrared light, configured to stimulate at least one of: mitochondrial activity, mitigate bacterial loads, regulate sebaceous gland activity, modulate inflammatory cytokines to avert inflammation, promote lymphatic drainage, boost cellular growth and immune responses and enhance cellular regeneration at the target anatomical region for accelerating surgical rehabilitation.

In another embodiment of the present disclosure, a method for accelerating surgical rehabilitation using the photobiomodulation device is disclosed. In the first step, the method includes positioning the irradiator configured with the plurality of treatment panels comprises the LEDs at a predefined distance from a target anatomical region using the adjustable expandable arm connected to the mainframe mounted on the mobile structure with the plurality of lockable wheels. In the next step, the method includes activating the photobiomodulation device through the user interface comprising the one or more clickable elements configured to control the one or more treatment parameters comprising at least one of: the preset and named treatment designed protocols selection, the wavelength selection, the irradiance, the energy delivered in per unit area, the emission mode, the irradiator configuration mode, one of: the flat mode and the curved mode of the irradiator, and one of: the sequential mode and the concurrent mode, at the time of selecting the multiple wavelengths, and the treatment time.

In the next step, the method includes determining, by the one or more sensors, at least one of: the target anatomical region temperature during operation and the distance between the irradiator and the target anatomical region to generate the temperature data and the distance data. In the next step, the method includes obtaining, by the one or more microcontrollers through the data obtaining subsystem, at least one of: the temperature data and the distance data from the one or more sensors in the real time. In the next step, the method includes regulating, by the one or more microcontrollers through the treatment control subsystem, the one or more treatment parameters based on at least one of: the temperature data and the distance data to emit at least one of: the red-light, the blue light, the yellow light, and the infrared light at the predefined wavelength.

In another embodiment of the present disclosure, a non-transitory computer-readable storage medium storing computer-executable instructions that, when executed by the one or more microcontrollers, cause the one or more microcontrollers to perform operations for accelerating surgical rehabilitation, the operations comprising: a) obtaining at least one of: the temperature data and the distance data from the one or more sensors associated with the photobiomodulation device in real time, b) triggering at least one of: the one or more alerts and the one or more termination commands of the irradiator associated with the photobiomodulation device if at least one of: the temperature data and the distance data falls outside the predefined threshold temperature data and the predefined operational distance range respectively, c) performing the comparative analysis between the real-time predefined wavelength of each LED of the plurality of LEDs and factory-calibrated reference wavelength for detecting output deviations in the predefined wavelength to calibration of each LED of the plurality of LEDs in each treatment panel of the plurality of treatment panels of the irradiator, d) logging rehabilitation session data including at least one of: timestamp, selected wavelengths, energy delivered per unit area, irradiance, emission mode, and sensor readings into one or more databases, and e) regulating the one or more treatment parameters based on at least one of: the temperature data and the distance data for emitting at least one of: the red-light, the blue light, the yellow light, and the infrared light at the predefined wavelengths by the plurality of LEDs. The emitted at least one of: the red-light, the blue light, the yellow light, and the infrared light, configured to stimulate at least one of: the mitochondrial activity, the mitigate bacterial loads, the regulate sebaceous gland activity, the modulate inflammatory cytokines to avert inflammation, promote lymphatic drainage, boost cellular growth and immune responses and enhance cellular regeneration at the target anatomical region for accelerating surgical rehabilitation.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limited in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIGS. 1A to 1G illustrate various views of a photobiomodulation device for accelerating surgical rehabilitation, in accordance with an embodiment of the present disclosure;

FIG. 1H illustrates a block diagram of the photobiomodulation device, in accordance with an embodiment of the present disclosure;

FIGS. 2A to 2U illustrate various embodiments of an interface associated with the photobiomodulation device for accelerating the surgical rehabilitation, in accordance with an embodiment of the present disclosure;

FIGS. 3A to 3C illustrate block diagrams associated with the photobiomodulation device for accelerating the surgical rehabilitation, in accordance with an embodiment of the present disclosure; and

FIG. 4 illustrates a flow chart of a method for accelerating the surgical rehabilitation using the photobiomodulation device, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

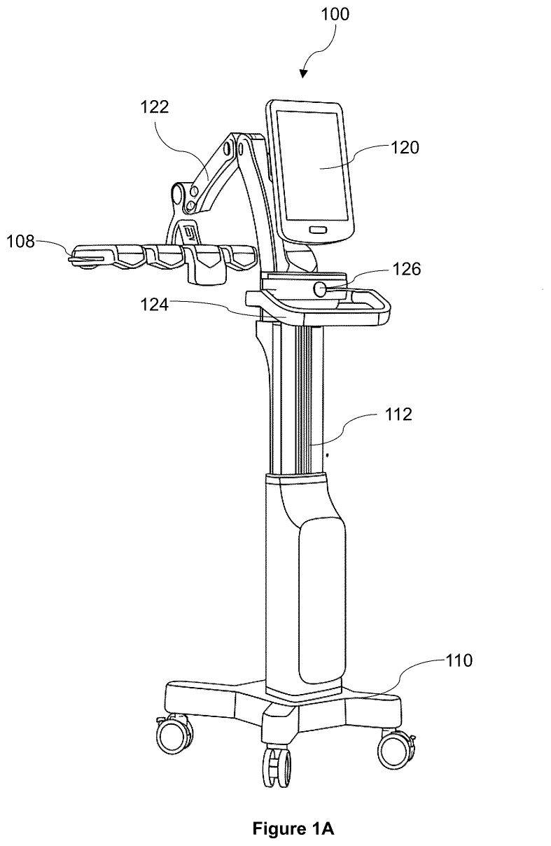

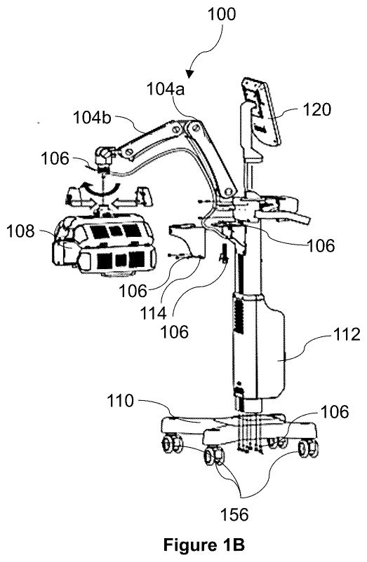

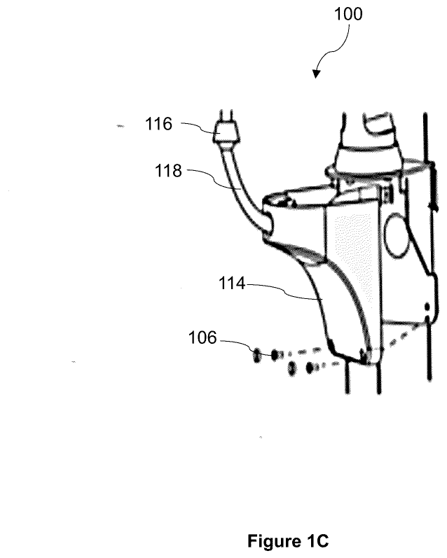

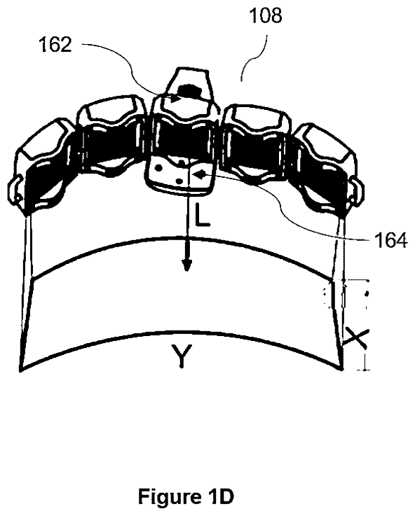

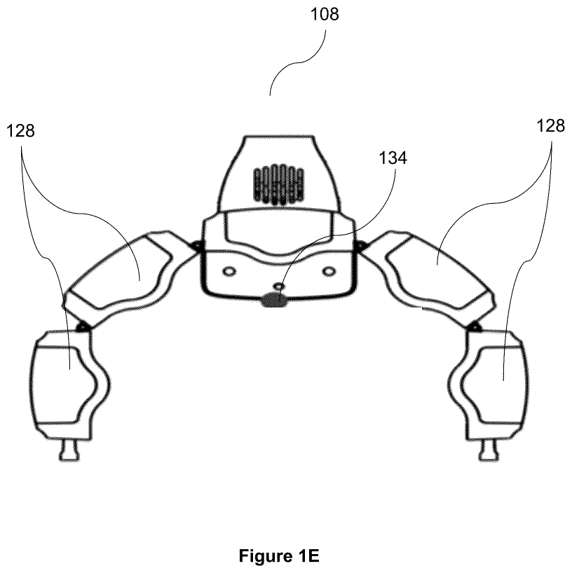

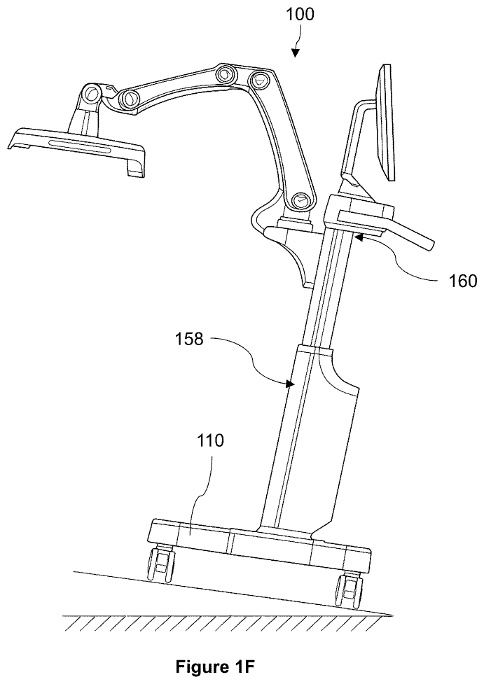

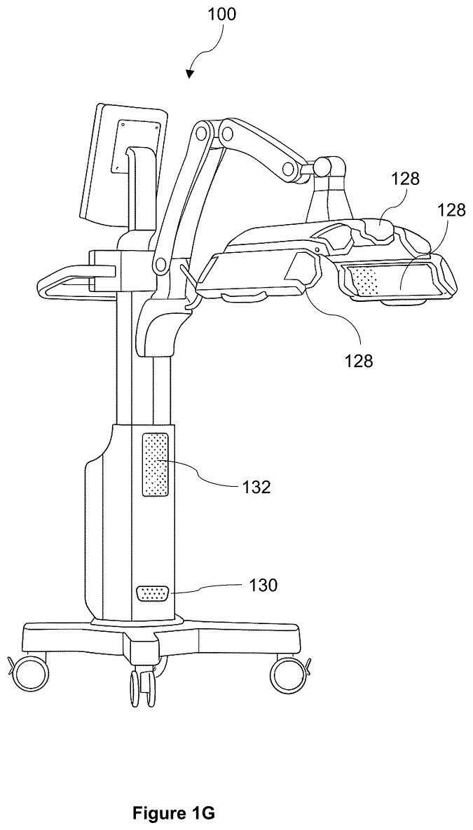

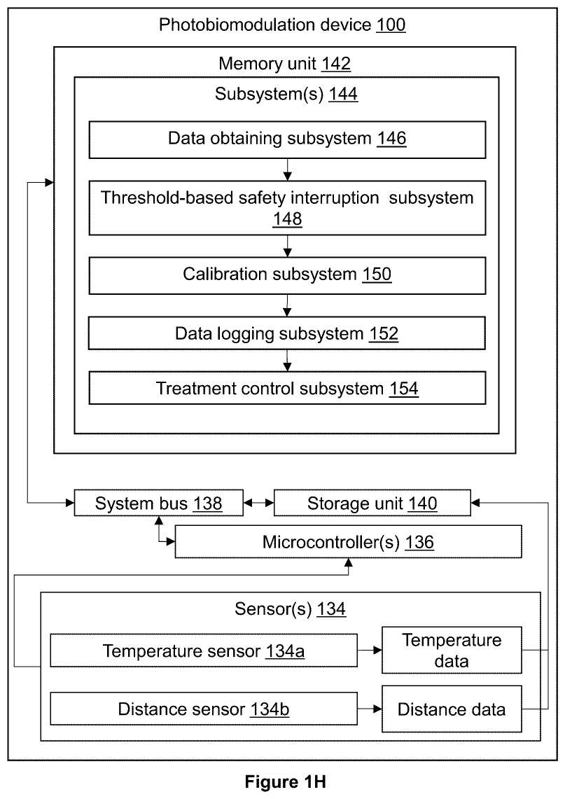

Referring now to the drawings, and more particularly to FIG. 1A through FIG. 4, where similar reference characters denote corresponding features consistently throughout the figures, there are shown preferred embodiments, and these embodiments are described in the context of the following exemplary system and/or method.

As used herein, the term “Photobiomodulation” describes an application of light at wavelengths ranging from 400 nanometers (nm) to 1100 nm to promote tissue healing, reduce inflammation, and induce analgesia. Emitted light energy is absorbed by cellular photoacceptors (chromophores), which are reported to mediate biological effects of the photobiomodulation.

FIGS. 1A to 1G illustrate various views of a photobiomodulation device 100 for accelerating surgical rehabilitation, in accordance with an embodiment of the present disclosure.

FIG. 1H illustrates a block diagram of the photobiomodulation device 100, in accordance with an embodiment of the present disclosure.

In an exemplary embodiment, the photobiomodulation device 100 (hereinafter referred to as the device 100) comprises a mobile structure 110, a mainframe 112, an adjustable expandable arm 122, an irradiator 108, one or more sensors 134, a user interface 120, and one or more microcontrollers 136. The device 100 is configured to activate several key mechanisms that change biological pathways of a skin. The device 100 is configured to improve Deoxyribonucleic Acid (DNA), Ribonucleic Acid (RNA) synthesis, and an immune system to repair the skin, facilitate new cell growth, and produce new collagen and elastin cells. The device 100 provides a versatile range of treatment modes, each tailored to address specific patient needs. The treatment modes may include, but not constrained to, at least one of: a thermal treatment, a detox treatment, a cellular repair treatment, a neoxcell treatment, a roxium treatment, and the like.

In an exemplary embodiment, the mainframe 112 is operatively mounted on the mobile structure 110 (i.e., base) with a plurality of lockable wheels 156. The mainframe 112 is configured to house at least one of: one or more electronic components and power sockets. The mainframe 112 is fixed to the mobile structure 110 with a plurality of fasteners 106. The one or more electronic components comprises the one or more microcontrollers 136, a memory unit, a mother board, a power supply module, one or more communication interface circuits, signal conditioning components, and one or more connectors for interfacing with the user interface 120, the one or more sensors 134, the irradiator 108, and the adjustable expandable arm 122. The mainframe 112 is also configured with a ventilation section 132 to ensure proper airflow and heat dissipation and a main power outlet 130 for reliable and efficient power supply. Further, the mainframe 112 is configured with a power button 126 to initiate and terminate the operation of the device 100.

In an exemplary embodiment, the irradiator 108 is configured with a plurality of treatment panels 128 operatively connected to the mainframe 112 on a first side 158 through the adjustable expandable arm 122. Each treatment panel 128 of the plurality of treatment panels 128 comprises the plurality of LEDs. The plurality of LEDs comprises 280 surface-mounted LEDs per each treatment panel 128 totaling 1400 LEDs for the plurality of treatment panels 128, arranged in a predefined matrix pattern. The plurality of treatment panels 128 comprises five treatment panels 128, each treatment panel 128 of the plurality of treatment panels 128 capable of being folded inward to conform to a curvature of one of: a human face and body contour. The plurality of LEDs configured to emit at least one of: red-light, blue light, yellow light, and infrared light. Each LED of the plurality of LEDs operate at a predefined wavelength.

In an exemplary embodiment, the predefined wavelengths of the red-light range between 623 nanometers (nm) and 643 nm. With the application of the red-light, fibroblast activity is increased, leading to enhanced collagen production. This strengthens an extracellular matrix, promotes tissue elasticity, and speeds up wound healing. Improved collagen synthesis ensures better scar outcomes, supporting skin regeneration and contributing to aesthetically pleasing results. The device 100 ensures effective detoxification, reduction of inflammation, and improved skin elasticity. The red-light is generally indicated to treat superficial, benign vascular, and pigmented lesions. The action of the device 100 on sebaceous glands decreases sebum production, which maintains a balanced skin environment. By reducing oiliness, the device 100 minimizes the chance of acne and other skin issues that may occur post-surgery, providing an additional layer of skin health support. The red-light treatment is delivered at a dose of 54 Joules/cm² over a 20-minute duration and is available as a preset and defined treatment protocol labeled “Thermal Treatment” in the user interface 120.

The predefined wavelengths of the blue light range between 407 nm and 427 nm. The blue light is configured to activate porphyrins in bacteria, generating reactive oxygen species (ROS). This process effectively kills the bacteria by damaging cell membranes, proteins, and the DNA, which lowers bacterial load on skin of one or more patients before surgery. By reducing the bacteria on the skin, the device 100 minimizes the risk of post-operative infections. The blue light is configured to reduce sebum production, minimizing infection risk. An energy infusion of the blue light is 60 Joules/cm² over 20 minutes. The blue light is generally indicated to treat dermatological conditions and specifically indicated to treat moderate inflammatory acne vulgaris. The blue-light treatment is delivered as the preset and defined treatment protocol labeled “Detox Treatment” in the user interface 120.

The predefined wavelengths of the yellow light range between 580 nm and 600 nm. The yellow light is configured to significantly lower pro-inflammatory cytokines (interleukin-6 (IL-6) and tumor necrosis factor-alpha (TNF-α)), which reduces swelling, bruising, and overall inflammation post-surgery. This mechanism speeds up tissue repair and promotes a quicker return to normal function. By modulating the inflammation, the device 100 also reduces pain associated with post-operative recovery, leading to a more comfortable healing process. The yellow light enhances mitochondrial activity, leading to increased Adenosine Triphosphate (ATP) production, which powers cellular repair and tissue recovery. The yellow light also stimulates nitric oxide release, promoting vasodilation and improved blood flow, which reduces swelling, bruising, and edema. The yellow light is generally indicated to treat dermatological conditions and specifically indicated for treatment of periorbital wrinkles and rhytides. The yellow-light treatment is delivered at a dose of 23 Joules/cm², and is available as the preset and defined treatment protocol labeled “Cellular Treatment” in the user interface 120.

In an exemplary embodiment, the device 100 stimulates a lymphatic system, increasing the efficiency of lymphatic drainage. By enhancing the ATP production and influencing nitric oxide pathways, the yellow light improves the contractility of lymphatic vessels, aiding in the removal of excess fluid and reducing edema. This is especially beneficial for preventing and alleviating swelling and fluid accumulation in surgical areas, which prolong recovery times.

The predefined wavelengths of the infrared light range between 825 nm and 845 nm. The infrared light is used for the temporary relief of at least one of: minor muscle and joint pain, arthritis, and muscle spasms, thereby relieving stiffness, promoting the relaxation of muscle tissue, and temporarily increasing local blood circulation where applied.

At least one of: the red-light, the blue light, the yellow light, and the infrared light is emitted in a pulsed mode with defined frequencies of 2 Hertz (Hz), 5 Hz, and 10 Hz at flashing interval times of 0.5 seconds(s), 0.2 s and 0.1 s, respectively. At least one of: the red-light, the blue light, the yellow light, and the infrared light is emitted in one of: a concurrent mode and a sequential mode, with multiple wavelength emission.

In an exemplary embodiment, the thermal treatment with the red light is used to boost cellular turnover, promoting a smoother and more youthful skin texture. The thermal treatment works by acting on ‘powerhouse’ cells in body cells called the mitochondria. With increased energy, the cells may perform the functions more efficiently, including repairing the skin, facilitating new cell growth, and producing new collagen and elastin cells. The thermal treatment stimulates cellular processes within the skin leading to various therapeutic benefits. The thermal treatment is effective for at least one of: superficial lesions, improved collagen production, improved appearance, pigmented lesions, and the like. The thermal treatment utilizes intense and focused red-light energy at a wavelength of 633 nanometers (nm). The energy infused is 54 J/cm², delivered over a 20-minute session, covering an effective surface area of 900 cm². This protocol is available as a preset and defined treatment protocol labeled “Thermal Treatment” in the user interface, enabling standardized, repeatable therapy for targeted skin rejuvenation applications.

In an exemplary embodiment, the detox treatment targets moderate inflammatory acne vulgaris. The blue light therapy is configured with a unique ability to penetrate the skin and reach the sebaceous glands where acne-causing bacteria, particularly Propionibacterium acnes, thrive. The detox treatment reduces the presence of harmful bacteria on the skin surface. This is beneficial for minimizing the risk of infection and creating a healthy skin microflora. The detox treatment also reduces the inflammation by suppressing the immune response of the skin. The detox treatment at least one of: enables pre-operative skin preparation, destroys acne causing bacteria, reduces the inflammation, and the like. The detox treatment effectively combats s various bacteria, including at least one of: the Propionibacterium acnes, Staphylococcus aureus (including MRSA), Pseudomonas aeruginosa, and Helicobacter pylori. The detox treatment exhibits a broad-spectrum antimicrobial effect by reducing bacterial load, minimizing infection risk, and promoting a healthy skin microflora.

The detox therapy utilizes intense and focused blue light energy at the wavelength of 417 nm. The energy infused is 60 J/cm² delivered in a 20-minute session. An effective surface area of irradiation is 900 cm². An antimicrobial action of the detox therapy is fundamentally tied to activating specific molecules within bacteria, particularly chromophores such as porphyrins leading to the production of reactive oxygen species (ROS).

The bacteria, unlike human cells, contain certain naturally occurring molecules known as the chromophores. One key chromophore group is the porphyrins. The porphyrins are photosensitive compounds, meaning the porphyrins absorb specific wavelengths of the light, especially in the blue spectrum (405 nm-470 nm). Different bacteria are configured with various types of porphyrins, such as protoporphyrin IX and coproporphyrin, which strongly absorb the blue light. When the detox therapy at a particular wavelength hits the chromophores, it excites electrons, thereby raising the electrons from a ground state to an excited state. Once the blue light is absorbed, the porphyrins undergo a process called photoexcitation. The photoexcitation occurs as photons from the blue light are absorbed by the electrons in a porphyrin molecule. This extra energy propels the electrons into a higher, unstable energy state. The excited electrons may not remain in the higher energy state indefinitely. As the electrons return to the ground state, the electrons release the absorbed energy in the form of molecular reactions. The molecular reactions lead to the formation of singlet oxygen (O₂) and other ROS, such as superoxide anions (O₂⁻) and hydroxyl radicals (OH). The singlet oxygen and other ROS are formed through a series of photochemical reactions triggered by the excited chromophores. The singlet oxygen is a highly reactive form of oxygen that carries significant cytotoxic properties. The primary ROS involved include at least one of: the singlet oxygen: highly reactive and directly damages lipids, proteins, and DNA, the superoxide anions: converted to hydrogen peroxide, which may lead to further oxidative damage, the hydroxyl radicals: extremely reactive and may cause extensive cellular damage, and the like.

The ROS, particularly the singlet oxygen and the hydroxyl radicals, attack the lipids in a bacterial cell membrane. This reaction, known as lipid peroxidation, results in the disruption of membrane integrity. The bacterial membrane, which acts as a barrier and a platform for cellular processes, becomes compromised, leading to increased permeability and loss of essential ions and molecules. The ROS also oxidizes bacterial proteins, damaging structural and functional components within the cell. The oxidation of enzymes and other proteins interferes with essential cellular functions, leading to reduced metabolic activity and impaired DNA repair mechanisms. The ROS may penetrate the bacterial cell and induce breaks in DNA strands. This leads to mutations and inhibits replication. The cumulative effect of DNA damage, protein inactivation, and membrane disruption ultimately incapacitates the bacterial cell. Due to membrane disruption, the bacteria lose the ability to maintain homeostasis, resulting in an uncontrolled influx and efflux of molecules. This disrupts ionic balance, which is crucial for energy production and cellular function. The oxidative stress caused by the ROS initiates bacterial cell death by interfering with metabolic pathways. Unlike the human cells, the bacteria lack a robust antioxidant defense system, making the bacteria more susceptible to oxidative stress. The combination of disrupted cellular processes, damaged membranes, and oxidative damage to cellular contents leads to bacterial cell lysis, where the cell essentially breaks apart and dies. The detox therapy is configured to lower the levels of pro-inflammatory cytokines such as interleukin-1 (IL-1) in the skin. Propionibacterium acnes (P. acnes) may induce the inflammation by triggering the cytokines, which signal the immune system to respond and exacerbate the inflammation around the sebaceous glands. By mitigating the inflammatory response, the detox therapy indirectly contributes to a reduction in the stress and inflammation experienced by the sebaceous glands. This translates to less swelling and discomfort, and in the long run, may normalize the production of sebum of the glands. In summary, the detox therapy initiates a cascade that begins with the excitation of the porphyrins, which leads to the formation of the ROS. The ROS then attacks various bacterial cell components, undermining the integrity and function of the cell until the cell ultimately succumbs to oxidative stress. This process is particularly effective in the bacteria due to the lack of advanced antioxidant defenses, setting the bacteria apart from mammalian cells, which are relatively more resilient to this light-induced oxidative damage. The detox therapy reduces bacterial load, oils, toxins, and prepares the skin for surgery, thereby lowering infection risk, minimizing complications, enhancing recovery, and promoting better scar outcomes.

In an exemplary embodiment, the cellular repair treatment with yellow light therapy increases cellular growth and boosts the immune system. The cellular repair treatment stimulates cellular activity, promoting tissue repair and regeneration, thus expediting the healing process by encouraging the natural repair mechanisms of the body. The cellular repair treatment provides significant benefits in reducing the appearance of scars, gradually making the scars less noticeable over time. The cellular repair treatment also aids in collagen production and tissue remodeling. The cellular repair treatment is configured to at least one of: reduce the inflammation, redness, bruising and swelling, reduce down-time post-surgical procedures, reduce down-time post laser procedures, reduce down-time post microneedling procedure, and the like.

The cellular repair treatment utilizes intense and focused yellow light energy at the wavelength of 599 nm. The energy infused is 23 J/cm² in 20 minutes. The effective surface area of irradiation is 900 cm². The cellular repair therapy at 590 nm effectively reduces bruising, swelling, and the edema by modulating the inflammation through the photobiomodulation. This process influences key signaling pathways, balancing cytokine production, reducing inflammation, enhancing tissue repair, and improving lymphatic function. The lymphatic system is a network of vessels and nodes that transports a lymph fluid throughout the body. The lymph fluid contains excess tissue fluid, immune cells, and waste products. Efficient lymph flow is essential for maintaining fluid balance, removing waste, and supporting immune function. The cellular repair therapy penetrates 0.5 nm to 2 mm in the skin and stimulates the cells within the lymphatic vessels. The cellular repair therapy particularly affects lymphatic endothelial cells (which line the vessels) and smooth muscle cells (which control vessel contraction). The cellular repair therapy enhances mitochondrial function, resulting in the increased ATP production. The additional energy supports cellular activities, such as protein synthesis and cell division, which are crucial for maintaining lymphatic vessel integrity and function. The cellular repair therapy also stimulates the release of nitric oxide (NO), a molecule that plays a significant role in vascular tone and fluid dynamics. The NO promotes vasodilation and reduces resistance within lymphatic vessels, allowing the lymph fluid to move more freely.

The lymphatic vessels are configured with smooth muscle cells that contract rhythmically to propel the lymph fluid through the lymphatic vessels. This process, known as lymphangiomotor activity, is critical for lymphatic drainage and is modulated by cellular energy levels and the function of smooth muscle cells. By increasing ATP availability, the cellular repair therapy supports the contraction of smooth muscle cells in the lymphatic vessels. With more ATP, the smooth muscle cells may sustain more effective and regular contraction-relaxation cycles. Improved contractility means that the lymphatic vessels may push the lymph fluid through the system more efficiently. Smooth muscle contraction is dependent on calcium signaling within the cells. The cellular repair therapy may influence calcium ion channels, enhancing the influx of calcium into the smooth muscle cells. The influx is crucial for initiating muscle contraction and maintaining rhythmic pumping activity that aids in fluid drainage. As the yellow light improves the lymphatic vessel contractions, lymphatic drainage increases. This enhanced lymphatic drainage clears excess fluid from the tissues, reducing the edema (swelling). By promoting the removal of the accumulated fluid due to injury and inflammation, the cellular repair therapy aids in faster recovery and lessens discomfort associated with fluid retention. The lymphatic vessels rely on the smooth muscle cells for propulsion of the lymph fluid. The smooth muscle cells contract in a coordinated manner, moving the lymph fluid against gravity and through various checkpoints such as lymph nodes, where the lymph fluid is filtered.

The increased cellular energy (ATP) resulting from yellow light exposure provides the energy needed for sustained muscle contraction. This is particularly important in the lymphatic system, where muscle contractions are not driven by a central pump such as a heart but rely on the vessel's own ability to contract rhythmically. The lymphatic vessels comprise one-way valves that prevent backflow, ensuring the lymph fluid moves in the correct direction. By enhancing the contractile strength of the smooth muscle cells, the cellular repair therapy supports valve function, as stronger and more coordinated contractions assist valves close properly, optimizing lymph movement. The inflammation and the injury lead to congestion within the lymph vessels. The cellular repair therapy reduces the inflammation around the vessels and promotes muscle cell activity, thereby overcoming obstructions that slow lymph fluid flow. Increased flow ensures that the lymph fluid, including any accumulated toxins and waste, is efficiently transported to lymph nodes for filtration and then back into the bloodstream. The nitric oxide is a signaling molecule that relaxes blood vessel walls, and the nitric oxide similarly affects the lymphatic vessels. By stimulating nitric oxide pathways, the cellular repair therapy relaxes the lymphatic vessels, which may reduce resistance and increase flow capacity. The release of the nitric oxide leads to vessel relaxation (vasodilation) within the lymphatic system, which may assist the lymph vessels in handling larger fluid volumes. This vasodilatory effect may increase the rate of lymph transport and reduce the likelihood of fluid buildup and the edema.

Key inflammatory cytokines, such as IL-6 and TNF-α, play essential roles in the body's response to injury. The key inflammatory cytokines are released by immune cells, including monocytes and macrophages, in reaction to tissue damage. Their primary functions include promoting vasodilation (dilation of blood vessels) and increasing capillary permeability, allowing the immune cells and fluids to move into the affected area. This process, although essential for an initial immune response, results in swelling and the fluid accumulates, and may be accompanied by bruising from blood leakage into surrounding tissues. The yellow light therapy reduces the production of the key inflammatory cytokines by influencing transcription and translation processes within the immune cells. The yellow light interacts with cellular photoreceptors, initiating signaling cascades that downregulate the production of the key inflammatory cytokines. By lowering IL-6 and TNF-α levels, the cellular repair therapy reduces blood vessel dilation and capillary permeability. This results in decreased fluid leakage into tissues, thereby minimizing swelling and edema. Both IL-6 and TNF-α are also involved in recruiting more immune cells to the injury site. Reducing the key inflammatory cytokines means fewer immune cells migrate to the area, further curbing the inflammation, swelling, and bruising.

A nuclear factor kappa B (NF-kB) is a pivotal transcription factor in the inflammatory response, regulating the expression of various pro-inflammatory genes, including those encoding IL-6, TNF-α, and other mediators. Upon activation, the NF-kB translocate into the nucleus of the immune cells, where the NF-kB promotes the transcription of inflammatory genes, amplifying the immune response and increasing inflammation. The cellular repair therapy inhibits the activation of the NF-kB, likely by altering intracellular signaling cascades through interactions with cellular photoreceptors.

By reducing NF-kB activity, the cellular repair therapy decreases the transcription of the pro-inflammatory cytokines, which in turn leads to a reduction in overall inflammation. The role of the NF-kB in the inflammatory cascade means that inhibiting the NF-kB prevents the propagation of the inflammation. By suppressing this pathway, the cellular repair therapy limits the chain reaction of cytokine release, thereby reducing the spread and intensity of swelling and the edema. The NF-kB also regulates adhesion molecules on the blood vessels, which are essential for the immune cells to adhere and migrate into the tissues. By downregulating the NF-kB, the cellular repair therapy reduces the capacity of the immune cells to infiltrate inflamed tissues, limiting further swelling and associated tissue damage. The inflammatory cytokines increase blood flow to an injured area, resulting in redness, warmth, and bruising. By reducing the IL-6 and the TNF-α production, and inhibiting the NF-kB, the cellular repair therapy controls blood flow to the area, mitigating the extent of bruising and speeding up the resolution of the edema. The cellular repair therapy facilitates the reabsorption of excess fluids accumulated in the tissues. Lower capillary permeability prevents ongoing fluid leakage, and the improved lymphatic function, bolstered by the photobiomodulation, aids in clearing away residual fluids and the waste products from the affected area. The smooth muscle cells in the lymphatic vessels contract rhythmically to move the lymph fluid. The cellular repair therapy improves the contraction-relaxation cycles by increasing ATP availability, allowing the smooth muscle cells to perform more efficiently. Additionally, the cellular repair treatment may influence calcium signaling within the smooth muscle cells, facilitating the contractions necessary for effective lymphatic drainage. The lymphatic vessels comprise the valves and the smooth muscles that regulate the flow and direction of the lymph fluid. The photobiomodulation increases a contractile strength of the smooth muscles, improving lymph circulation and clearing waste products from inflamed tissues.

Beyond reducing the pro-inflammatory cytokines, the cellular repair therapy also modulates anti-inflammatory cytokines such as IL-10, which are essential for resolving the inflammation. The cellular repair therapy promotes the polarization of macrophages toward an M2 (reparative) state, which supports healing. The yellow light influences IL-17, which is involved in tissue repair, creating a balanced environment that aids recovery without exacerbating inflammation. Chronic inflammation may result from an imbalance of the cytokines, leading to prolonged inflammatory states and potential tissue damage. By promoting IL-10 and controlled IL-17 production, the cellular repair therapy assists the body shift from an inflammatory state to a healing state, supporting homeostasis and preventing the chronic inflammation. Fibroblasts are specialized cells in the connective tissue that play a crucial role in wound healing and the tissue repair. The fibroblasts produce collagen and other components of the extracellular matrix (ECM), which provides structural support and elasticity to the tissues. The cellular repair therapy penetrates the skin and interacts with cellular photoreceptors in the fibroblasts. This interaction triggers signaling pathways that stimulate fibroblast proliferation and activity. The energy absorbed by the fibroblasts enhances the activity of specific receptors and enzymes that promote cellular growth and collagen synthesis. For instance, the cellular repair therapy upregulates transforming growth factor-beta (TGF-β), a key regulator of collagen production. With increased fibroblast activity, the synthesis of collagen is boosted, leading to enhanced ECM formation. Collagen fibers provide the framework for new tissue, promoting tissue strength, flexibility, and repair. This is particularly important in wound healing, where new tissue needs to be formed to replace damaged areas.

The cellular repair therapy stimulates the mitochondria, the energy centers within the cells, by activating cytochrome c oxidase, an enzyme in the electron transport chain. This activation leads to the increased production of ATP, which is the primary energy carrier within the cells. With more ATP available, the cells are configured with greater energy resources to carry out metabolic activities, including protein synthesis, DNA repair, and cellular replication. This accelerated metabolism supports overall cell function and aids in the repair of damaged tissues. The increased ATP production from the cellular repair therapy supports the synthesis and release of growth factors, such as vascular endothelial growth factor (VEGF), fibroblast growth factor (FGF), and epidermal growth factor (EGF). The growth factors are essential in tissue repair and regeneration. The VEGF, for instance, promotes angiogenesis (the formation of new blood vessels), which is essential for delivering the oxygen and nutrients to heal tissues. This process ensures that newly formed tissue is well-supported by an adequate blood supply, facilitating faster and more effective tissue repair. The growth factors such as the EGF and the FGF stimulate the proliferation and migration of skin cells, including keratinocytes and the fibroblasts. The skin cells migrate to the site of injury and contribute to wound closure and regeneration of the epidermis and dermis layers, reinforcing tissue integrity. The cellular repair therapy reduces excessive production of the pro-inflammatory cytokines (e.g., IL-6 and TNF-α), which may lead to the chronic inflammation and tissue damage. By lowering the pro-inflammatory cytokines, the cellular repair therapy maintains a more balanced immune response. While reducing the inflammation, the yellow light also boosts the anti-inflammatory cytokines such as IL-10, which resolve the inflammation and promote a transition from an inflammatory to a healing environment. This cytokine balance prevents prolonged inflammation and creates conditions conducive to the tissue repair.

The cellular repair therapy stimulates macrophages to adopt a reparative phenotype (M2 macrophages), which are involved in the tissue repair and clearance of cellular debris. The cellular repair therapy also supports the activity of lymphocytes, which play a role in both innate and adaptive immune responses. By enhancing macrophage function, the cellular repair therapy improves the removal of dead cells and other debris from the injury site. This cleanup process is essential for creating a clean environment where new tissue may grow and thrive. With a balanced immune response and enhanced activity of the immune cells, cellular repair treatment protocol prevents infections that may otherwise compromise the tissue repair. The improved immune function ensures that the body's defenses are optimized to protect the healing area, allowing for more efficient and complete recovery. The cellular repair therapy alleviates post-surgical inflammation, leading to reduced pain and faster recovery. The cellular repair therapy improves lymphatic drainage, minimizes fluid retention. The cellular repair therapy stimulates cellular activity and promotes collagen production, thereby speeding up the healing process and improving skin quality. The cellular repair therapy enhances circulation and improves hydration and elasticity, contributing to better skin texture post-surgery.

In an exemplary embodiment, the roxium treatment combines the blue light and the yellow light therapy. The roxium treatment actively reduces oxidative stress by promoting the body's natural antioxidant defenses, ultimately safeguarding cells from harm and enhancing overall cellular health. As the lymphatic system becomes more efficient at removing impurities and waste, the skin may appear clearer and less prone to blemishes, such as acne and breakouts. A reduction in toxins contributes to a healthier complexion. The roxium treatment minimizes the appearance of enlarged pores, which are caused by excessive sebum production. The excessive sebum on the skin may hinder the effectiveness of skincare products by forming a barrier. Lowering the sebum production enhances the absorption of the skincare products, making the skincare products more effective. The roxium treatment reduces oxidative stress by promoting the body's natural antioxidant defenses. This protects the cells from damage and improves overall cellular health.

The roxium treatment utilizes a combination of the blue light at 417 nm and the yellow light at 599 nm. The energies infused are 60 J/cm² and 23 J/cm² respectively over 20 minutes. The effective surface area of irradiation is 900 cm². The roxium treatment combines the benefits of the detox treatment and the cellular repair treatment to provide a powerful and synergistic treatment, targeting both oxidative stress and skin health at multiple levels promoting optimal recovery and long-term aesthetic results. The detox treatment stimulates the production of antioxidants in the cells, neutralizing harmful reactive oxygen species (ROS) generated by environmental factors, protecting cells from oxidative damage and reducing inflammation. The cellular repair treatment enhances blood circulation, improving oxygen and nutrient delivery, further supporting the skin's natural antioxidant defenses. The detox treatment targets the sebaceous glands, reducing oil production, which minimizes post-operative breakouts and oily skin. The cellular repair treatment complements this by reducing the inflammation and supporting lymphatic function, further minimizing the appearance of enlarged pores, and promoting overall clearer skin health. This dual action ensures the body's waste removal processes are maximized, reducing swelling post-operatively. The roxium treatment provides a fortified body's natural anti-oxidants defenses. The roxium treatment reduces post-operative skin breakouts and improves skin quality and texture. The roxium treatment provides better aesthetic outcomes and enhanced scarring profile.

In an exemplary embodiment, the neoxcell treatment combines the red-light treatment and the yellow light therapy. The neoxcell treatment ensures structural support, elasticity, and firmness, while also preventing pigmented lesions for a uniform complexion. The neoxcell treatment assists the lymphatic system in eliminating waste and excess fluid more efficiently, thereby reducing post-surgery fluid retention. Including the fibroblasts, secreting collagen proteins that maintain the structural framework of the tissues, aiding in efficient cellular repair and promoting more efficient and improved healing. By targeting and encouraging more collagen synthesis and remodeling may provide the structural support to the skin and maintain elasticity and firmness. By balancing melanin production, the neoxcell treatment may reduce the likelihood of hyper-pigmentation occurring after the surgery, resulting in progressive fading and a more uniform complexion.

In an exemplary embodiment, the neoxcell treatment utilizes a combination of the yellow light at 599 nm and the red light at 633 nm. The energies infused are 23 J/cm² and 54 J/cm² respectively over 20 minutes. The effective surface area of irradiation is 900 cm². The neoxcell treatment at least one of: combines the benefits of the cellular repair treatment and the thermal treatment energies, promotes comprehensive skin rejuvenation, accelerates recovery, and enhances post-surgical healing by providing synergistic effects. The cellular repair therapy penetrates the superficial skin layers, activating the lymphatic system and stimulating the smooth muscle cells in the lymphatic vessels. This increased lymphatic flow and drainage removes toxins, waste products, and excess fluids from the tissues, thereby reducing post-operative swelling and edema. The thermal treatment penetrates deeper into the skin, stimulating fibroblast activity and enhancing blood circulation. This promotes collagen production, which is essential for maintaining skin structure, elasticity, and firmness. The improved blood flow complements the lymphatic drainage process initiated by the cellular repair treatment, ensuring that the waste products are efficiently removed and promoting an optimal environment for healing. The cellular repair treatment balances the melanin production, reducing the risk of hyperpigmentation. This ensures a more uniform complexion, preventing pigmented lesions following the surgery. The neoxcell treatment provides efficient detoxification and enhanced aesthetic results.

In an exemplary embodiment, for a pre-surgical acne optimization tool, in an acne severity index, grade 1 indicates mild severity. Clinical findings state open and closed comedones with few inflammatory papules and pustules. Grade 2 indicates moderate severity. The clinical findings state the papules, and the pustules are mainly on the face of the one or more patients. Grade 3 indicates Moderate-Severe severity. The clinical findings state multiple papules and pustules, and occasional inflamed nodules, also on a chest and a back of the one or more patients. In the detox treatment, the blue light penetrates the acned skin, and the detox treatment initiates a series of chemical reactions that generate the singlet oxygen, a highly reactive toxic form of oxygen, that kills the bacteria. The detox treatment may destroy the acne-causing bacteria. The detox treatment is configured to at least one of: kill the acne causing bacteria (P. acnes), reduce the inflammation, reduce sebaceous hyperplasia for preventing future breakouts, assist calm irritated skin, reduce redness associated with the acne, and the like.

In an exemplary embodiment, for a pre-surgical eczema optimization tool, in the detox treatment, the blue light penetrates the skin and induces an anti-inflammatory and anti-proliferative effect. The blue light is beneficial for hyperproliferative and chronic inflammatory atopic dermatitis. The blue light is configured with anti-Inflammatory effects. The blue light is configured to reduce itching sensation and improve skin barrier function. The blue light is configured with antibacterial properties, which may be beneficial in preventing and treating secondary skin infections that may occur in eczema-damaged skin.

In an exemplary embodiment, a pre-surgical rosacea optimization protocol in a rosacea severity index, a mild grade indicates a score of 2. The clinical findings state few small papules and pustules, mild erythema. A moderate grade indicates the score of 3. The clinical findings state one of: several small papules/pustules and large papules/pustules, and moderate erythema. A severe grade indicates the score of 4. The clinical findings state numerous small and/or large papules/pustules, and severe erythema. The cellular repair with the yellow light penetrates the skin to enhance circulation and red blood cell production, targeting sun damage, a primary contributor to rosacea. The yellow light functions at the cellular level through a process called the photobiomodulation, thereby altering the biological pathways of the skin. The cellular repair is configured with anti-Inflammatory and soothing effects. The cellular repair is configured to at least one of: stimulate the blood circulation, provide healing and rejuvenation, by assisting in repair damage, especially that caused by exposure to Ultraviolet (UV) rays, a known trigger for rosacea flare-ups, reduce visibility of blood vessels, and the like.

In an exemplary embodiment, the adjustable expandable arm 122 operatively connected to the mainframe 112 on the first side 158 via a baffle 114 with the plurality of fasteners 106. The adjustable expandable arm 122 is provided for supporting and positioning the irradiator 108. The adjustable expandable arm 122 comprises two metal longitudinal members (104a and 104b) namely a first longitudinal member 104a and a second longitudinal member 104b, operatively connected by a vertically movable joint.

The first longitudinal member 104a, having a length of approximately 17.5 inches, is mounted at one end to a supporting frame located at the first side 158 (a top rear section) of the mainframe 112. This mounting is accomplished via a horizontally rotatable base secured with a pair of metal pins, thereby allowing rotational or pivotable motion in the horizontal plane. The first longitudinal member 104a provides horizontal adjustability and positional control of the expandable arm assembly 122 and the attached irradiator 108.

The second longitudinal member 104b, having a length of approximately 11 inches, is pivotally connected at one end to the distal end of the first longitudinal member 104a via the vertically movable joint. The opposite end of the second longitudinal member 104b is connected to a dual-axis (vertical and horizontal) movable joint, which is configured to support and adjustably position the irradiator 108.

The adjustable expandable arm 122 is configured to provide at least one of: vertical direction and horizontal direction to the irradiator for reaching a target anatomical region. The adjustable expandable arm 122 is configured to rotate positive 80 degrees and negative 80 degrees from a longitudinal axis of the mainframe. The adjustable expandable arm 122 is configured to tilt positive 15 degrees and negative 15 degrees from a pivotal point of the adjustable expandable arm 122.

In an exemplary embodiment, the user interface 120 is operatively mounted on the mainframe 112 at a first end 160 (i.e., a top end of the mainframe 112), which corresponds to a top end of the mainframe 112. The user interface 120 is configured to allow the user to control and adjust one or more treatment parameters relevant to the operation and performance of the device during therapeutic procedures. The one or more treatment parameters comprise at least one of: preset and named treatment designed protocols selection, wavelength selection, irradiance, energy delivered in per unit area, emission mode, irradiator configuration mode, one of: a flat mode and a curved mode of the irradiator, and one of: a sequential mode and a concurrent mode, at a time of selecting multiple wavelengths, and treatment time.

The user interface 120 is configured to provide at least one of the following types of input mechanisms to facilitate user control: one or more clickable elements, one or more voice comment inputs, one or more touchscreen inputs, one or more gesture-based controls, one or more remote-control inputs, and one or more programmable physical buttons. These input mechanisms allow the user to interact with the device and adjust one or more treatment parameters with precision and flexibility, depending on user preference or clinical requirements.

Specifically, the user interface 120 is configured to comprise at least one of the following control elements: a) a wavelength selector for choosing one or more wavelengths to be emitted by the irradiator 108, enabling the delivery of specific optical energies tailored to therapeutic objectives, b) a power level selector for regulating the irradiance delivered to the target anatomical region, allowing for intensity adjustments based on tissue sensitivity and treatment protocol; c) a treatment duration timer for setting the treatment time, ensuring controlled exposure in line with predefined therapy schedules; d) a treatment dosage input for setting a dose of the energy delivered in per unit area (J/cm²), facilitating precise dosing of optical energy based on area-specific requirements; e) a mode selector for switching between pulsed mode and continuous mode, providing flexibility in energy delivery methods; f) a treatment controller for one of: initiating treatment, terminating treatment, and pausing treatment, g) an irradiator mode selector for switching between one of: the flat mode and a curved mode of the irradiator 108, accommodating anatomical variations and treatment region contours; h) a treatment mode selector for switching between one of: the sequential mode and the concurrent mode, at the time of selecting multiple wavelengths, and treatment time, and i) a preset protocol selector for applying the predefined treatment parameters based on one of: a treatment type and a body region, streamlining the setup process and ensuring adherence to standardized therapeutic protocols.

In the illustrative embodiment, the user interface 120 comprises a Liquid Crystal Display (LCD) screen 102 having a diagonal dimension of 12.1 inches, which is configured to provide full operational control. The LCD screen 102 displays treatment parameters, user inputs, system status, and other operational indicators in real time, serving as both an interactive control surface and an information display for the user. The configuration of the user interface 120 as described enables any person skilled in the art to understand, configure, and operate the device in accordance with treatment requirements. It ensures compatibility with a range of clinical scenarios while offering flexibility in control schemes and operational modes.

In an exemplary embodiment, the user interface 120 assemble with the mainframe 112 by aligning with a notch and connecting the line between the user interface 120 and the mainframe 112, with the plurality of fasteners 106 used, and hole plugs assembled (2 upper plugs and 2 lower plugs) at a first side (back side). Further, a LEMO cable 118 from the user interface 120 is pass through a circular hole on the baffle 114, plug in the LEMO cable 118 (as shown in FIG. 1C), install the baffle 114 back to the mainframe 112 and fix the baffle 114 with the plurality of fasteners 106, and install a rubber ring 116 on the LEMO cable 118 into the circular hole in the baffle 114. A cantilever frame 124 is operatively connected on top of the mainframe 112, which is configured to adjust UP, DOWN, LEFT and RIGHT the device 100 as needed. The capacity of the cantilever frame 124 is 70 Newtons (N).

In an exemplary embodiment, the one or more sensors 134 are operatively positioned on one or more treatment panels 128 of the plurality of treatment panels 128. The one or more sensors 134 are configured to determine at least one of: target anatomical region temperature during operation and distance between the irradiator and the target anatomical region for generating temperature data and distance data. The one or more sensors 134 enable the user to monitor critical operational parameters that directly affect treatment efficacy and safety. Specifically, the temperature data allows for precise regulation of thermal exposure on the treated tissue of the target anatomical region, while the distance data ensures the irradiator is positioned within an optimal range for energy delivery. This configuration provides the user or the control device with feedback for adjusting treatment parameters dynamically, based on real-time sensor readings.

The one or more sensors 134 comprises at least one of: a temperature sensor 134a and a distance sensor 134b configured to generate temperature data and distance data, respectively, which is stored in a storage unit 140 for further processing by the one or more microcontrollers 136. The temperature sensor 134a is embedded at a second side 164 of the central treatment panel of the plurality of treatment panels 128 and is specifically configured to determine the target anatomical region temperature during treatment operation. This sensor aids in preventing overheating of the tissue and helps maintain therapeutic temperatures within a safe and effective range. Additionally, the distance sensor 134b is integrated at a third side 162 of the central treatment panel of the plurality of treatment panels 128. The distance sensor 134b is configured to determine the distance between the irradiator and the target anatomical region, thereby ensuring proper positioning of the irradiator 108 relative to the patient's body surface. This feature is essential for consistent energy distribution, especially in modalities requiring specific standoff distances for wavelength penetration and intensity control. This arrangement of the one or more sensors 134 not only enhances the ability of the device 100 to monitor and adjust its performance in real-time but also contributes to treatment safety, reproducibility, and precision. By embedding and integrating the temperature sensor 134a and the distance sensor 134b within specific sides of the central treatment panel, the device 100 enables a closed-loop feedback mechanism for optimizing therapeutic output.

In an exemplary embodiment, the memory unit 142 is operatively connected to the one or more microcontrollers 136 via a system bus 138. The system bus 138 functions as the central conduit for data transfer and communication between the one or more microcontrollers 136, the memory unit 142, and the storage unit 140. The system bus 138 facilitates the efficient exchange of information and instructions, enabling the coordinated operation of the device 100.

The memory unit 142 comprises a set of computer-readable instructions in form of a plurality of subsystems 144. The memory unit 142 is configured to be executed by the one or more microcontrollers 136. The plurality of subsystems 144 comprises a data obtaining subsystem 146, a threshold-based safety interruption subsystem 148, a calibration subsystem 150, a data logging subsystem 152, and a treatment control subsystem 154.

In an exemplary embodiment, the data obtaining subsystem 146 is configured to obtain at least one of: the temperature data and the distance data from the one or more sensors or from the storage unit 140 in real time for data processing. The data obtaining subsystem 146 operates under the control of the one or more microcontrollers 136 and is responsible for continuously monitoring and acquiring sensor-generated signals during operation of the device 100. The data obtaining subsystem 146 may include analog-to-digital converters (ADCs), signal amplifiers, digital buffers, or embedded firmware routines that format and timestamp the incoming data. The data obtaining subsystem 146 is configured to function in real time, meaning it provides the most current sensor values without processing delay, thereby enabling dynamic and responsive regulation of treatment parameters such as irradiance, energy delivered per unit area, and emission mode.

In an exemplary embodiment, the threshold-based safety interruption subsystem 148 is configured to trigger at least one of: one or more alerts and one or more termination commands of the irradiator 108 if at least one of: the temperature data and the distance data falls outside a predefined threshold temperature data and a predefined operational distance range, respectively. The threshold-based safety interruption subsystem 148 operates in conjunction with the data obtaining subsystem 146 and the one or more microcontrollers 136 and is responsible for monitoring the real-time operational safety of the device 100. The threshold-based safety interruption subsystem 148 continuously compares the temperature data and distance data acquired from the one or more sensors 134 against predefined reference limits stored in the storage unit 140. The predefined threshold temperature data corresponds to a maximum permissible surface temperature at the target anatomical region beyond which photothermal injury, discomfort, or skin damage may occur. Similarly, the predefined operational distance range represents the allowable proximity between the irradiator 108 and the target anatomical region, ensuring accurate irradiance levels and proper light distribution during therapy.

When either the temperature data exceeds the threshold temperature data or the distance data falls outside the predefined operational distance range, the threshold-based safety interruption subsystem 148 initiates a safety response. The response comprises triggering at least one of: the one or more alerts and the one or more termination commands. The one or more alerts may include audible alarms, visual indicators on the user interface 120, or both, to notify the operator of a deviation from safe operating conditions. The one or more termination commands automatically disable the LEDs within the irradiator 108, halting light emission until corrective action is taken or parameters return within acceptable limits.

In some embodiments, the one or more alerts may include real-time sensor readouts or error codes displayed on the user interface 120, enabling the operator to identify the specific cause of the safety interruption. The termination of the irradiator 108 is performed through a hardware or software-level signal issued by the one or more microcontrollers 136 to the LED driver circuits, effectively cutting off the output without affecting other system functions.

In an exemplary embodiment, the calibration subsystem 150 is configured to calibrate each LED of the plurality of LEDs by performing a comparative analysis between a real-time predefined wavelength and a factory-calibrated reference wavelength for detecting output deviations in the predefined wavelength. The calibration subsystem 150 functions as a precision control module within the device 100 and is operatively executed by the one or more microcontrollers 136 using calibration routines stored in the storage unit 140.

The plurality of LEDs are configured to emit at least one of: red-light, blue light, yellow light, and infrared light at predefined wavelengths. During prolonged use, factors such as LED aging, thermal stress, and power supply fluctuations may cause slight deviations in the actual emitted wavelength and associated irradiance from the predefined wavelength. These deviations, if left uncorrected, may compromise treatment accuracy and clinical efficacy. Therefore, the calibration subsystem 150 is provided to ensure consistent optical output performance over the operational life of the device 100. The calibration subsystem 150 operates by initiating a calibration cycle, either automatically after a predetermined cumulative usage time (e.g., 500 hours) or manually through user initiation via the user interface 120. During this cycle, the real-time predefined wavelength emitted by each LED of the plurality of LEDs is measured using an internal or external wavelength detection mechanism, such as an optical sensor, spectrometer, or a calibrated photodiode array. The real-time predefined wavelength is then compared against the factory-calibrated reference wavelength values stored in the storage unit 140. The factory-calibrated reference wavelength represent the optimal wavelength output determined during the manufacturing process under controlled environmental conditions. The calibration subsystem 150 performs a comparative analysis for each LED or LED group to detect output deviations between the measured and reference wavelengths.

If deviations are detected, the calibration subsystem 150 computes the necessary correction factors and applies them to a LED driver circuitry, thereby adjusting one or more of: the LED current, the pulse width modulation (PWM) duty cycle, or activation timing to realign the emitted wavelength with the factory-calibrated reference wavelength. The calibration subsystem 150 may also log calibration data, including time of calibration, deviation magnitudes, and correction parameters into the storage unit 140 for auditability and maintenance tracking. In certain embodiments, the calibration subsystem 150 may also trigger an alert through the user interface 120 if the deviation exceeds a predefined tolerance limit, indicating that manual inspection or LED replacement may be required. This proactive calibration mechanism ensures the device 100 continues to deliver consistent and effective light-based therapy tailored to the predefined wavelengths necessary for stimulating mitochondrial activity, modulating inflammation, promoting lymphatic drainage, and enhancing cellular regeneration.

In an exemplary embodiment, the data logging subsystem 152 is configured to log rehabilitation session data, including at least one of: a timestamp, selected wavelengths, energy delivered per unit area, irradiance, emission mode, and sensor readings into one or more databases or the storage unit 140. The data logging subsystem 152 operates under the control of the one or more microcontrollers 136 and is implemented as part of the computer-readable instructions stored in the memory unit 142. The data logging subsystem 152 serves a critical role in maintaining a verifiable, retrievable, and secure record of each therapeutic session delivered by the device 100. The data logging subsystem 152 records the timestamp associated with the initiation, execution, and termination of each rehabilitation session. This timestamp includes both date and time data for traceability and compliance with clinical documentation standards. The selected wavelengths refer to the specific light outputs such as the red-light, the blue light, the yellow light, and the infrared light, chosen by the user or automatically selected based on a preset protocol stored in the device 100. The energy delivered per unit area (J/cm²) is calculated based on the product of irradiance and treatment time and is a critical metric in ensuring the correct therapeutic dose is administered. Irradiance refers to the power delivered per unit area (e.g., in mW/cm²), and may vary in real time depending on factors such as distance to the treatment site and surface contour. The emission mode, whether continuous or pulsed, and in the case of pulsed mode, the specific frequency used (e.g., 2 Hz, 5 Hz, or 10 Hz), is also logged as part of the session profile. The sensor readings logged by the data logging subsystem 152 include at least one of: temperature data from the temperature sensor 134a, and distance data from the distance sensor 134b. These readings are important for post-session safety validation, treatment analysis, and for diagnosing any irregularities in device 100 behavior or patient response. The one or more databases into which the rehabilitation session data is logged may reside in the storage unit 140 or on an external storage medium accessible via communication interface circuits. In some embodiments, the data logging subsystem 152 may support periodic data exports to external systems such as hospital record servers, cloud-based storage platforms, or electronic medical records (EMR) software, using standard communication protocols. In a pulse mode, the device 100 operates by just turning “ON” and “OFF” at a fixed rate to give the skin some rest between the intervals, and the device 100 may not operate closely to common pulsed lasers. Effective irradiance of the irradiator 108 is adjusted according to the treatment needed. The device 100 is configured with a double switch protection of power switch and power-on password. The device 100 does not cause any damage to the skin cells.

In an exemplary embodiment, the treatment control subsystem 154 is configured to regulate the one or more treatment parameters based on at least one of: the temperature data and the distance data for emitting at least one of: the red-light, the blue light, the yellow light, and the infrared light at the predefined wavelengths. The treatment control subsystem 154 functions as an active, closed-loop control module that receives continuous input from the data obtaining subsystem 146 and the one or more sensors 134, and makes real-time adjustments to ensure optimal therapeutic delivery by the device 100.

The one or more treatment parameters regulated by the treatment control subsystem 154 include at least one of: wavelength selection, irradiance, energy delivered per unit area (J/cm²), emission mode (continuous or pulsed), and treatment duration. These parameters are adjusted based on the real-time values of the temperature data and the distance data to ensure that energy is delivered safely, accurately, and uniformly across the target anatomical region. For instance, if the distance between the irradiator 108 and the treatment site increases, the treatment control subsystem 154 may automatically increase the treatment duration or irradiance level to maintain the target energy delivered per unit area.

The treatment control subsystem 154 is further configured to dynamically regulate treatment duration based on the real-time irradiance values and the selected energy delivered per unit area. This allows the device 100 to ensure that the total light dose (in J/cm²) delivered to the tissue is consistent, regardless of variations in LED performance, sensor data, or operator adjustments. The subsystem calculates the required treatment time by dividing the desired energy delivered per unit area by the measured irradiance and automatically terminates the session once the calculated energy has been fully administered.

The emitted at least one of: the red-light, the blue light, the yellow light, and the infrared light is delivered at predefined wavelengths optimized for specific biological effects. These effects include, but are not limited to: stimulating mitochondrial activity to enhance ATP production; mitigating bacterial loads, particularly with blue light for superficial pathogens; regulating sebaceous gland activity to improve dermal balance; modulating inflammatory cytokines to avert inflammation and reduce postoperative swelling; promoting lymphatic drainage to decrease edema and facilitate fluid removal; boosting cellular growth and immune responses for faster tissue remodeling; and enhancing cellular regeneration through fibroblast proliferation and angiogenesis.

Each wavelength contributes selectively and synergistically to the acceleration of surgical rehabilitation. For example, red-light and infrared light penetrate deeper into tissue to promote vascularization and tissue regeneration, while yellow light and blue light address surface-level conditions such as inflammation and microbial presence. The treatment control subsystem 154 ensures the correct combination, timing, and intensity of these light outputs based on the patient's anatomical region, sensor feedback, and selected protocol.

Specifications of the device 100 comprises: a) an effective irradiation area: 900 centimeters (cm)²±10%, b) irradiation distance: 6 cm±1 cm, c) an irradiator configured with five treatment panels 128 each equipped: 280 Surface Mount Device-Light Emitting Diode (SMD-LED), d) display mode: the LCD screen 102, e) 110V powered, standard for any office: 100-240 Volts, 50/60 Hertz (Hz), f) input power: 600 volt-amperes (VA), g) fuse specification, model & rating: T8.0AL/250V φ5*20, h) dimensions: 490 millimeters (mm)×470 mm×1370 mm, i) weight: 87 pounds (lbs)/39.5 kg, and j) structure: wheeled.

Features of the device 100 may comprise, but not restricted to, at least one of: 1400 Medical-Grade High Power SMD-LED Chips, no consumables and maintenance for lifetime of 50,000 hours, 12.1″ user interface 120 combined with humanized Graphics User Interface (GUI) design to make the operation more concise and smoother with the ability to rotate 90 degrees horizontally and 30 degrees vertically, the irradiator 108 combined in one treatment head, unique dose mode and radiation intensity calibration to ensure precision of the treatment, five programmable treatments settings allows saving customized times and doses, an artificial intelligence (AI) temperature control system and high temperature automatic fuse for safer treatment, an infrared sensor measurement to ensure accurate measurement of working distance, a real-time display and early warning to ensure the accuracy of treatment, a secondary optical configuration for the SMD-LEDs to make the irradiator 108 more balanced, a live intensity detection function, and the like.

The device 100 is configured with the adjustable expandible arm 122 attached to the plurality of treatment panels 128 that may rotate 160 degrees left and right. The plurality of treatment panels 128 may be one of: curved and flattened as per a treated surface. An intensity control ranges from 1 percent to 100 percent. A time control ranges from 1 minute to 99 minutes, and a dose control ranges from 1 J/cm²-99 J/cm². The device 100 is configured to: provide the infrared sensor measurement to ensure accurate measurement of working distance, provide real-time display and early warning to ensure the accuracy of treatment, provide live intensity detection function, that may be controlled, and provide the secondary optical configuration for the SMD-LEDs to make the irradiator 108 more balanced.

The error between the effective irradiance and the nominal value is less than 25 percent, and the effective irradiance is less than 200 mW/cm². The effective irradiance of the red-light ranges from 20 milliwatts (mW)/cm² to 96 milliwatts (mW)/cm². The effective irradiance of the blue light ranges from 10 mW/cm² to 120 mW/cm². The effective irradiance of the yellow light ranges from 5 mW/cm² to 35 mW/cm². The effective irradiance of the infrared light is ≤70 mW/cm². The effective irradiance of the red light/infrared light ranges from 20 mW/cm² to 166 mW/cm². The effective irradiance of the blue light/infrared light ranges from 10 mW/cm² to 190 mW/cm². The noise generated by the device 100 is less than 60 decibels (dB) A-weighted (A). Under normal working condition, a stand of the device 100 is adjusted up, down, left, and right, and the pair of irradiators may be fixed at any angle. An irradiance collection probe (as shown in FIG. 1E) is configured with precision instruments.

The device 100 is configured with a timer with an error not greater than ±2 percent of a set value, and continuous working time ≤99 min. The function of manually stopping radiation output is realized through the software pauses and stop buttons. The pair of irradiators may be replaceable. The five treatments may be preset. The device 100 is configured with a calibration function. The device 100 is configured with a time mode. The device 100 is configured with a dose mode. The device 100 is configured with two functions continuous output and pulse output. The operating environment of the device 100 must meet the following requirements: temperature: 5 degrees Celsius to 35 degrees Celsius, relative humidity: ≤85 percent, and atmospheric pressure: 700 Hectopascal (hPa) to 1060 hPa.

A maximum power density of the red light is 96 mW/cm 2. The maximum power density of the blue light is 120 mW/cm 2. The maximum power density of the yellow light is 35 mW/cm². The maximum power density of the infrared light is 70 mw/cm². Standard doses in Joules of the red light are 155 J/cm². The standard doses in Joules of the blue light are 144 J/cm². The standard doses in Joules of the yellow light are 42 J/cm². The standard doses in Joules of the infrared light are 84 J/cm².

Technical specifications of the device 100 are shown in Table 1.

TABLE 1

| | Light Source | | | | | | | | |
|---|---|---|---|---|---|---|---|---|---|
| | Red | Blue | Red | Blue | Yellow | Red | Blue | Yellow | Infrared |
| Wavelength | 633 | 417 | 633 | 417 | 590 | 633 | 417 | 590 | 850 |
| (nm) | | | | | | | | | |
| LED | 700 | 700 | 467 | 467 | 466 | 350 | 350 | 350 | 350 |
| Quantity | | | | | | | | | |
| (PCS) | | | | | | | | | |
| Light | 75 | 80 | 52 | 55 | 25 | 40 | 42 | 20 | 33 |
| Intensity | | | | | | | | | |
| (mW/cm²) | | | | | | | | | |
| Light Power | 78.4 | 126 | 52.3 | 84 | 26 | 39.2 | 63 | 19.6 | 31.5 |
| (W) | | | | | | | | | |
| Total LED | 1400 | | 1400 | | | 1400 | | | |
| Quantity | | | | | | | | | |
| (PCS) | | | | | | | | | |
| Total Light | 204.4 | | 162.3 | | | 153.3 | | | |
| Power (W) | | | | | | | | | |

Before treatment, the one or more patients should wash the treatment area, and the one or more surgeons and the one or more patients should wear special goggles to prevent possible irradiator 108 damage to the eyes. It is recommended that irradiation should be provided for 20 minutes each time.

The device 100 is operated by following steps: a) install the required irradiator 108, pull the handles on both sides of the irradiator 108 to adjust the irradiator 108 to a suitable working arc. The working arc of the irradiator 108 may be adjusted from a flat surface to a semi-circular arc, b) press a button to connect through a power supply, the indication light and the LCD screen 102 will light up, indicating the working mode is entered, c) check whether the device 100 works normally, d) the effective irradiation area of the irradiator 108 is shown in the FIG. 1D, should be calculated according to an expression expressed in Equation 1.

$$S = ( X - 1 ) * ( Y - 1 ) * 5 \qquad Equation 1$$

The patient must wear special protective goggles and place the irradiator 108 at a position that is 6 cm±1 cm from the treatment area of the one or more patients, e) one of: select the irradiation mode, set the color, treatment time/dose, and irradiance, and directly invoke one of the therapies, f) press a play button is operatively positioned in the exact middle of the function buttons on the user interface 120 in the LCD screen 102, configured to start the treatment, g) To pause or suspend the treatment, press a pause button located to a left of the play button on the user interface 120. This action of the pause button temporarily turns off the irradiator 108 without resetting the treatment time. The session can be resumed from the point at which it was paused, h) the time returns to zero, the irradiator 108 goes out, and the one or more alerts sounds, indicating that the treatment is over, and i) To stop the treatment entirely, press a stop button located to the right of the play button on the user interface 120. This action of the stop button immediately terminates the entire treatment and resets the treatment time to 20 minutes and the dose to the default value of the selected treatment protocol, further, the power supply is still connected.

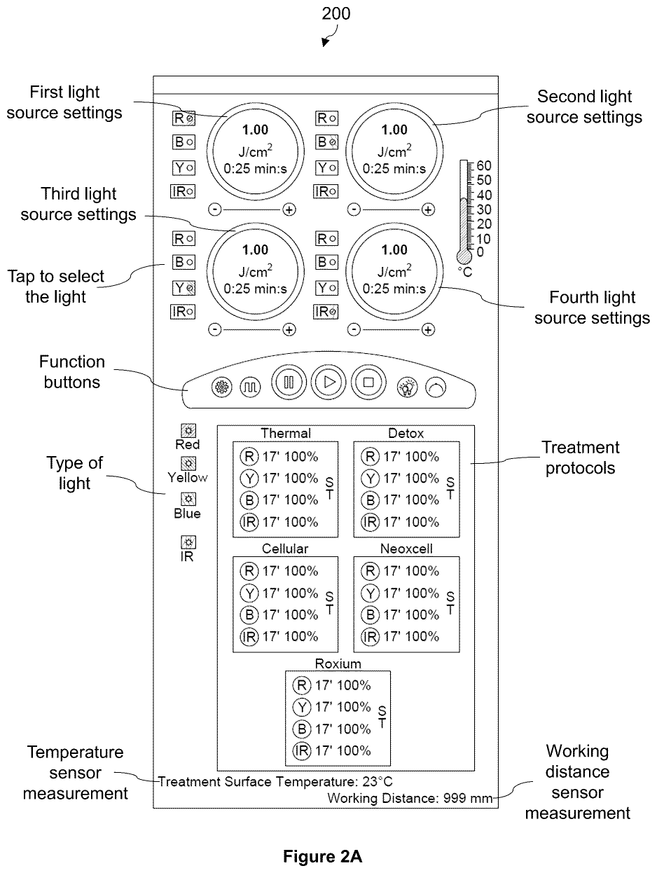

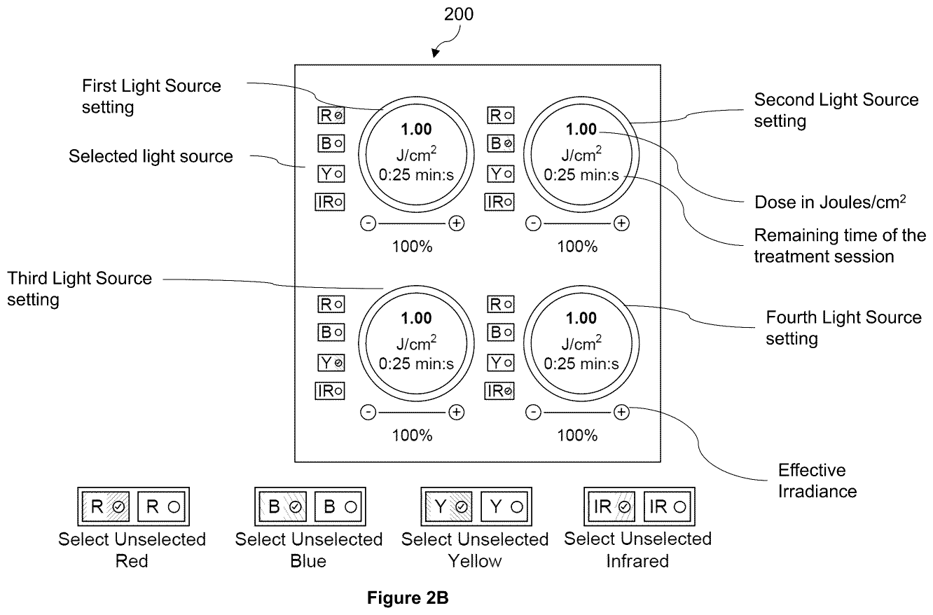

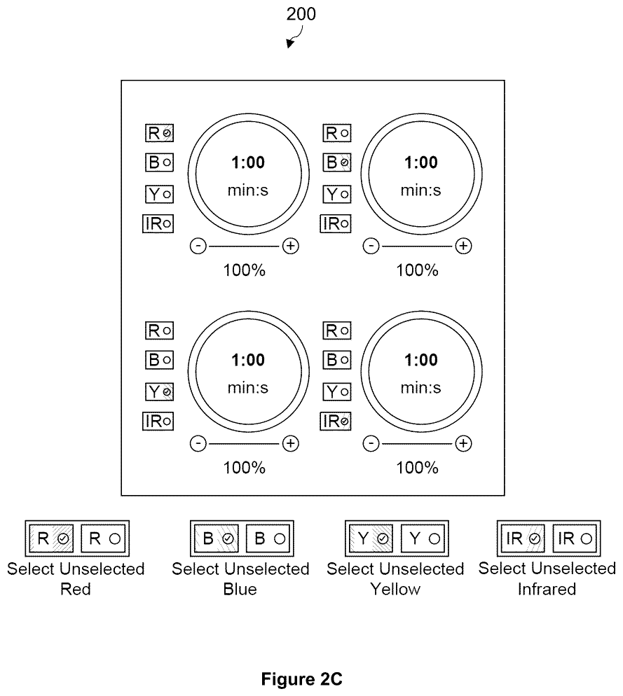

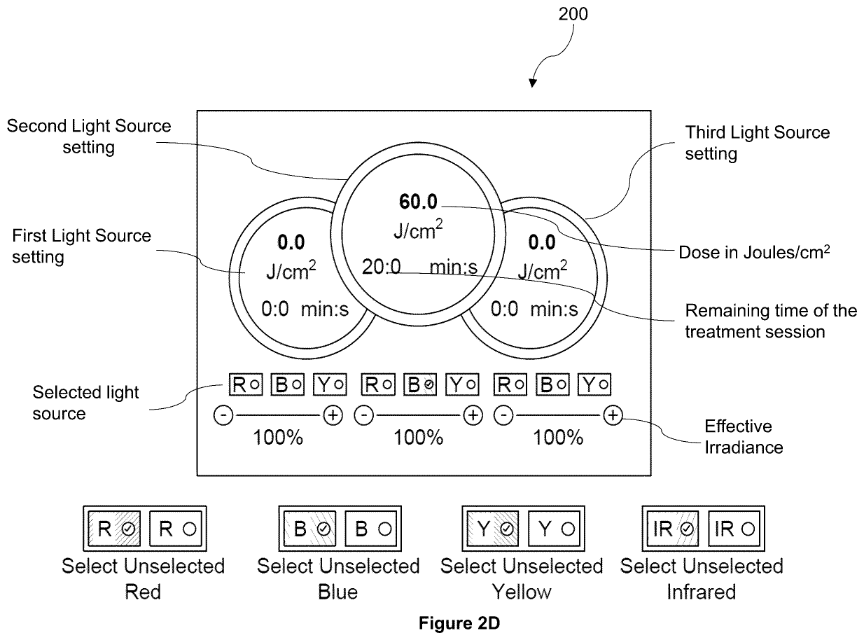

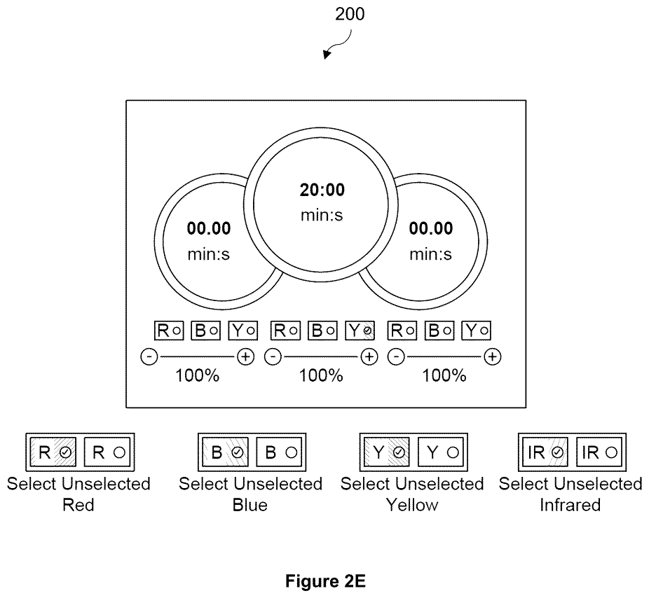

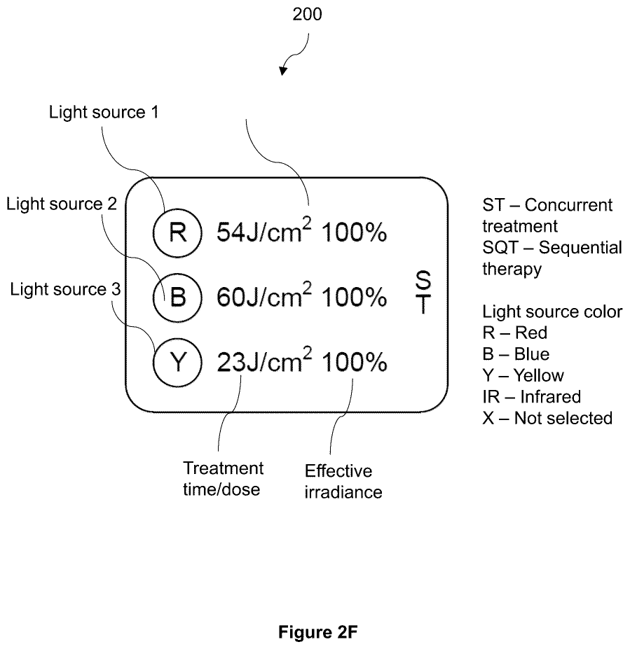

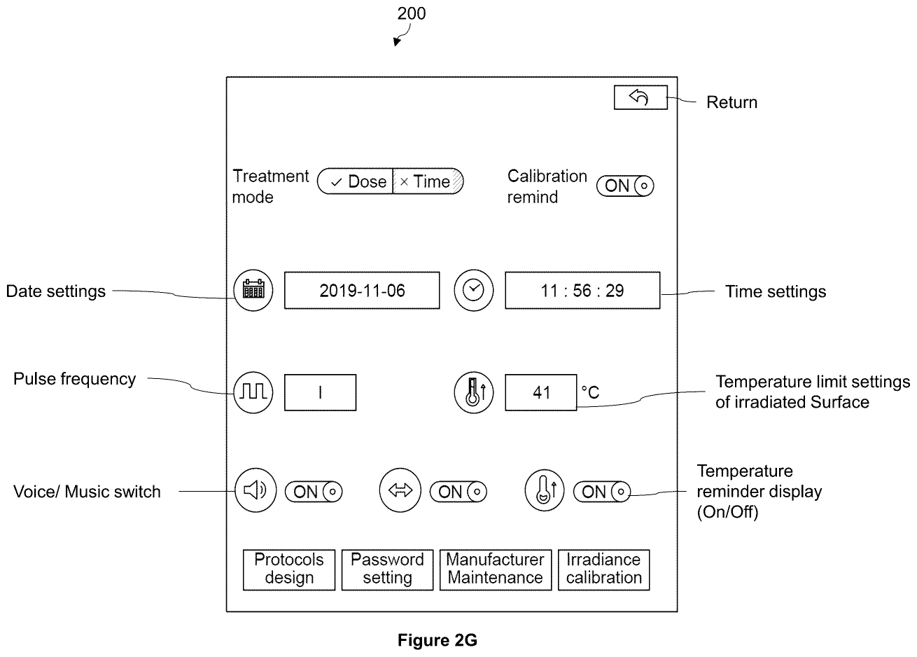

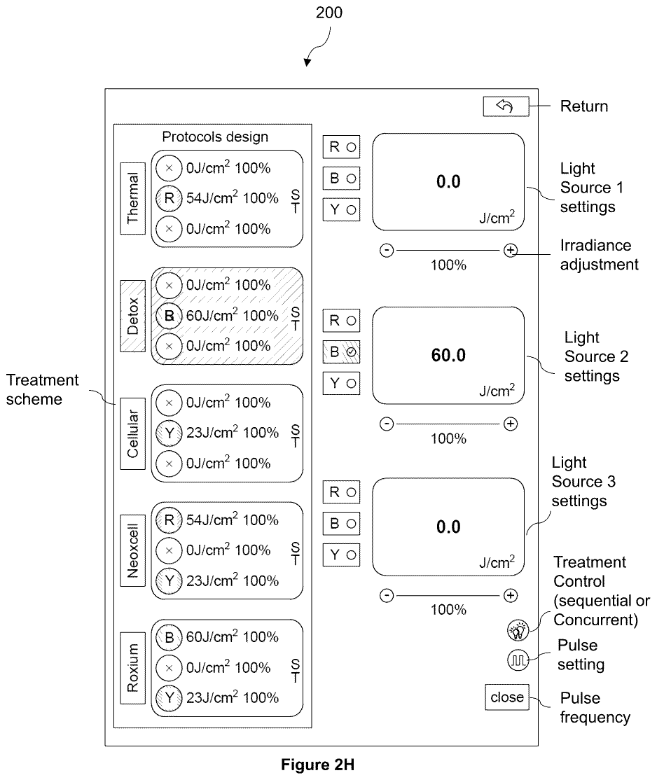

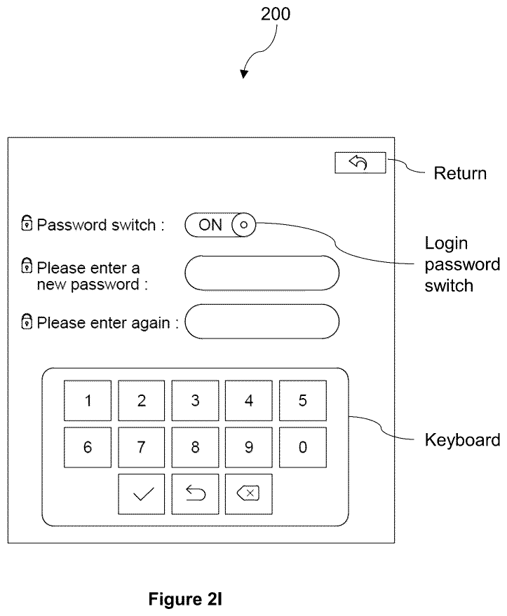

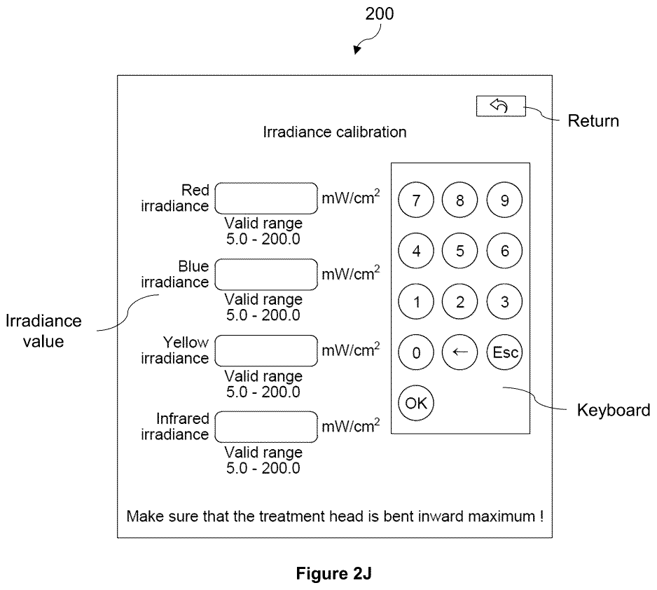

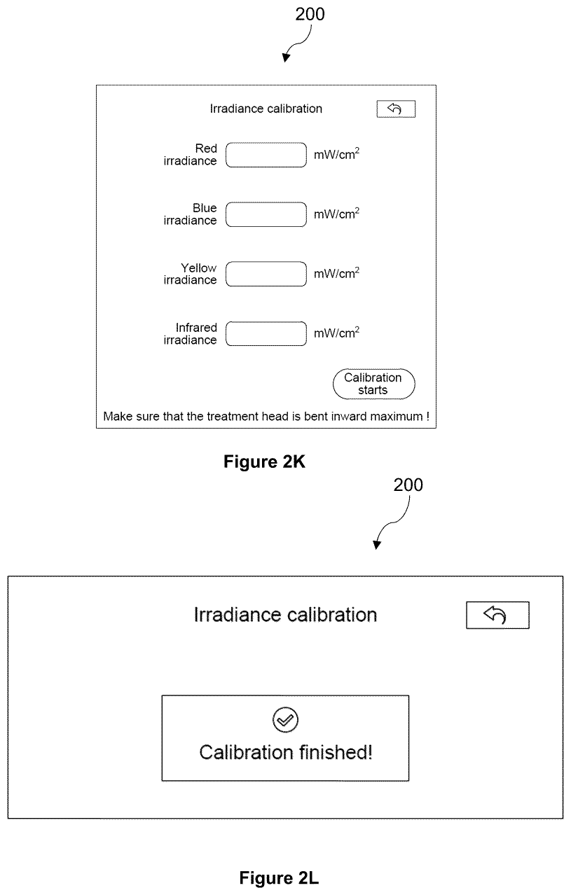

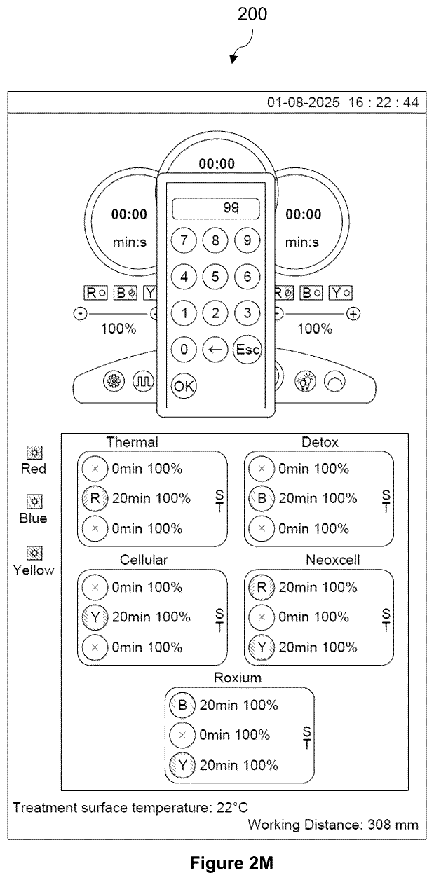

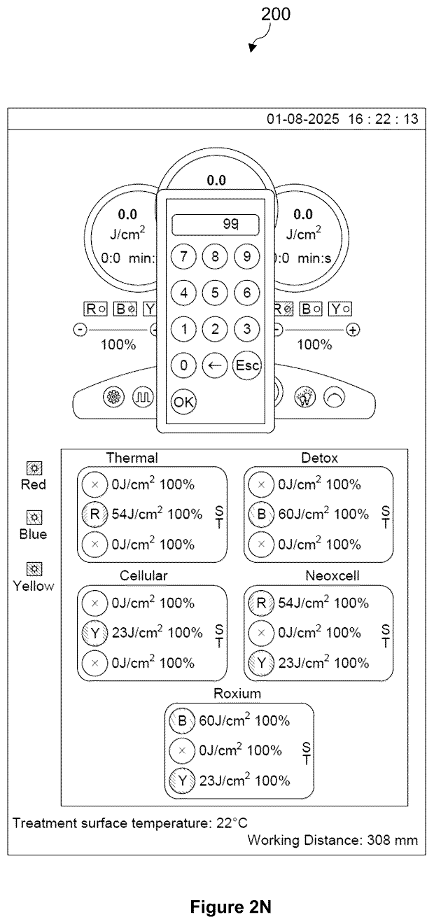

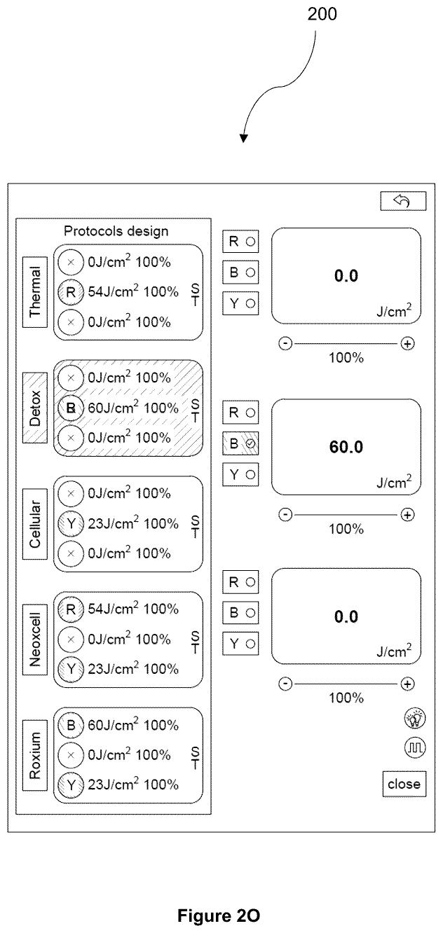

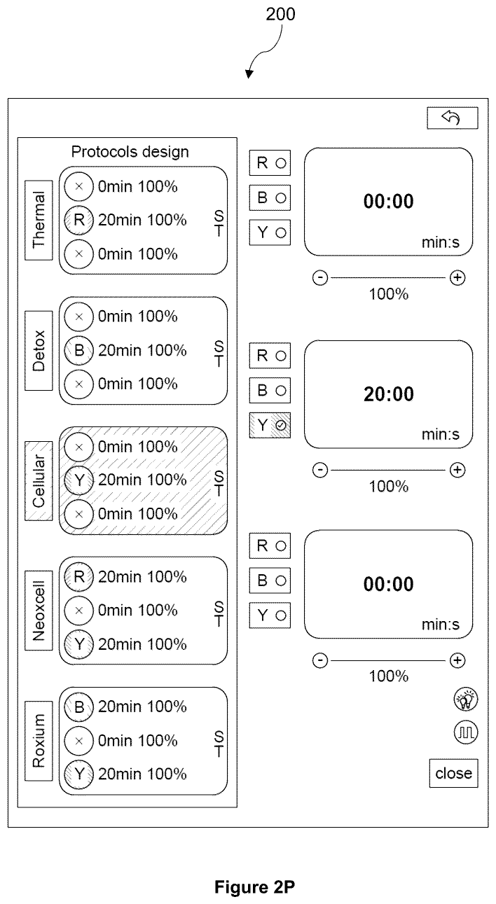

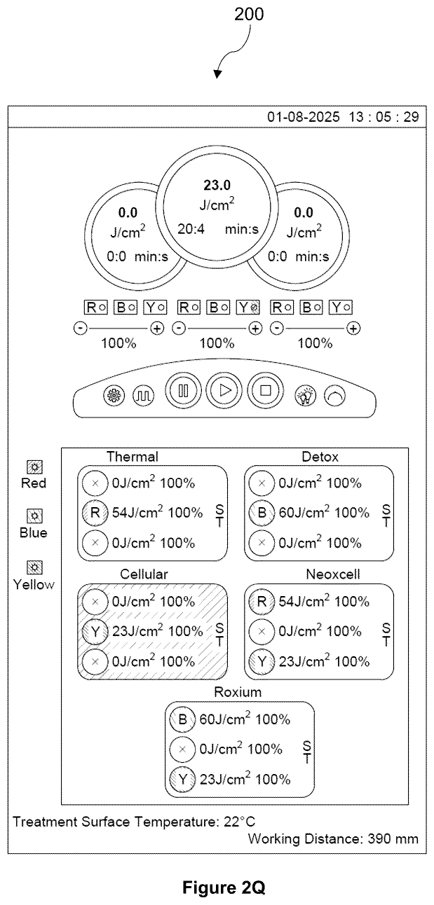

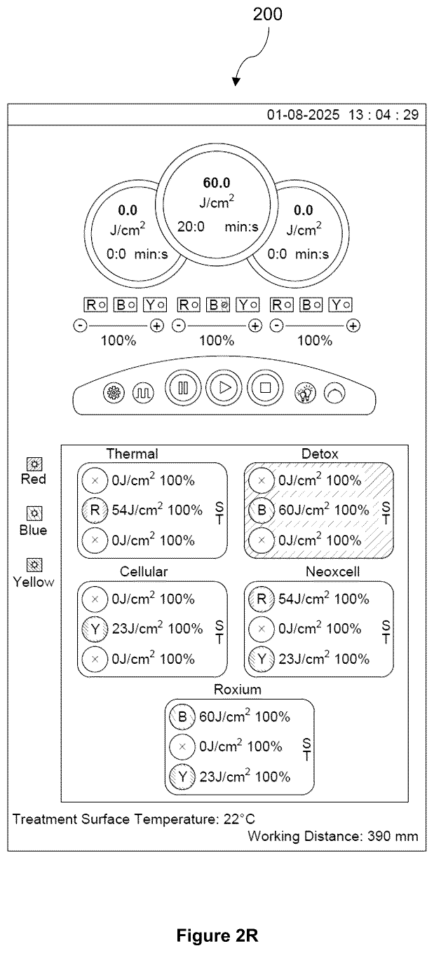

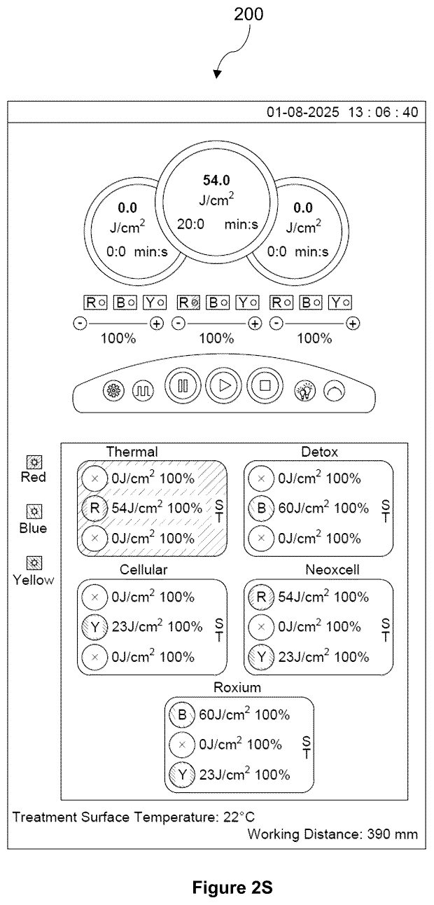

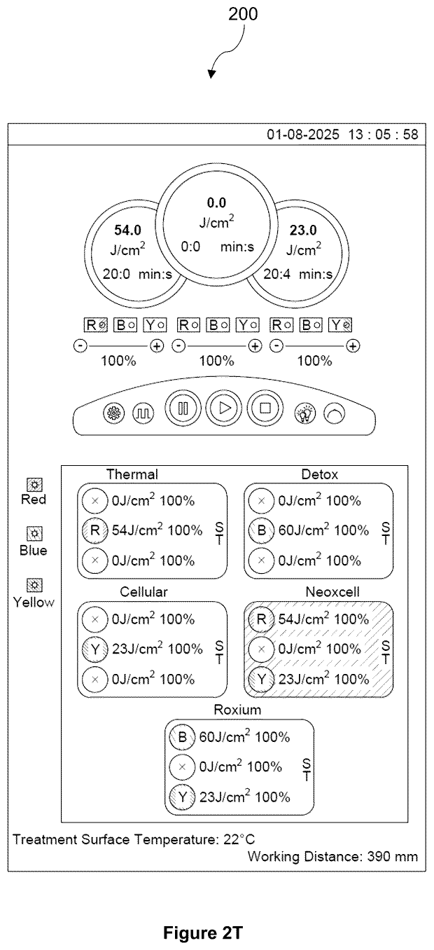

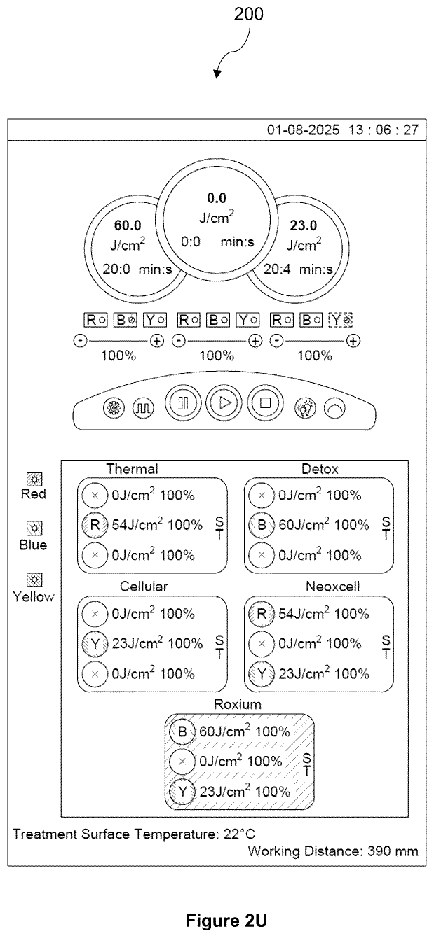

FIGS. 2A to 2U illustrate various embodiments of an interface 200 associated with the device 100 for accelerating the surgical rehabilitation, in accordance with an embodiment of the present disclosure.

In an exemplary embodiment, a window on the interface 200 is displayed as the “Exposure time window” or “Irradiation dose window” according to the selected “treatment mode”. Time/dose and irradiance may be set for each treatment panel 128. The color should be set for mixed irradiator 108; “R” is the red light, “B” is the blue light, “Y” is the yellow light, “IR” is the infrared light. The same color of light may not be selected simultaneously in several windows of the irradiator 108 setting. When no treatment scheme is selected, the window allows direct modification of the number to set time or dose. Click the number, a numeric keypad may appear, input the required value, and click OK to save the setting. This position shows the remaining time or dose during the treatment. Directly click on a position in the irradiance progress bar or press “+” or “−” to adjust the source irradiance. Press “+” or “−” once, the number increases or decreases by 1 percent. Pressing and holding “+” or “−” may cause continuous digital adjustment. When restarted, the device 100 may automatically call the last treatment mode and settings when it is turned off.

Five preset treatment schemes are displayed in the “Treatment scheme” on the main interface. Click on a scheme, the selected scheme will turn to the green button, and the preset treatment time/dose, effective irradiance, the irradiator 108 color, and treatment mode corresponding to the selected scheme may be called in the current main interface. A treatment mode allows to switch between “Time mode” and “Dose mode” (as shown in FIG. 2G), and the main interface (as shown in FIG. 2A) and treatment scheme interface may be displayed according to the selected treatment mode. As depicted in FIG. 2G, a calibration reminder switch allows to select “switch ON” or “OFF”, the automatic calibration reminder function. If the one or more surgeons select “ON”, the device 100 may remind the user to perform automatic calibration of the effective irradiance after 500 hours' use of the irradiator 108, to ensure the correctness of the irradiator 108 output. Click the “Set Date” or “Set Time” window, and the “+,−” buttons appear at both ends of the date or time, press “+” indicates the number plus 1, and press “−” indicates the number minus 1. Pressing and holding the “+” or “−” button may realize continuous digital adjustment. By clicking a pulse frequency button, the pulse flashing frequency may switch among I (2 Hz), II (5 Hz), and III (10 Hz). In the set temperature limit, click on the “Set Temperature Limit” window, the “+,−” buttons appear at both ends. Press “+”, the number plus 1, and press “−”, the number minus 1. Pressing and holding “+” or “−” may realize continuous digital adjustment. The range is 35-60. A voice switch button allows the voice to switch between ON and OFF states. A working distance button enables to select “Switch ON” or “OFF” the working distance reminder. If the one or more surgeons select “ON,” the working distance may be displayed at the bottom of the main interface. Temperature of the irradiated surface button allows to switch “ON” or “OFF”, the irradiated surface temperature reminder. If the one or more surgeons select “ON,” the temperature of the irradiated surface may be displayed at the bottom of the main interface. Press a “Scheme Design” button in a setting interface (as shown in FIG. 2G). Choose one of the schemes and set one of: the treatment time/dose, effective irradiance, the irradiator 108 color and treatment mode according to the “Light Source Setting” on the main interface. A password setting button in the setting interface (as shown in FIG. 2G) is configured to turn the login password switch “ON” or “OFF”.

Irradiance calibration (“Manual calibration” and “Automatic calibration”) may be selected according to different device 100 configurations. For the manual calibration: enter the irradiance of the irradiator 108 according to the result of manual calibration. Click the input field to be corrected, the keyboard may pop up. Enter the corrected value and click “OK” to save the correction result. For automatic calibration: bend the irradiator 108 inward to the most, click “Start Calibration”, the device 100 may automatically calibrate the irradiance of each treatment panel 128 of the plurality of treatment panels 128 in the irradiator 108. When the calibration is completed, click “Calibration finished” to end the calibration. A manufacturer maintenance button is used for maintenance of the device 100 by the manufacturer and not applicable to the patient and the user.

Analysis and troubleshooting methods of common faults of the device 100 are listed in Table 2. The interface 200 (as shown in FIG. 2M) between the device 100 and the user allows to choose the therapy time, the pulse mode, start or stop therapy, and turn on or turn off the device 100.

TABLE 2

| SN | Fault description | Possible causes | Troubleshooting method |
|---|---|---|---|
| 1 | The power switch is | Failure of power | Check whether the power |
| | switched on, but there | supply system. | cord connection is loose and |
| | is no display in the | | whether the fuse is |
| | display window. | | damaged. |
| | | Control circuit | Notify the manufacturer or |
| | | failure. | local agent. |
| 2 | Light source does not | The time set or | Reset the time and restart. |
| | work. | startup procedure is | |
| | | incorrect. | |
| | | Loose connector | Retighten the connector |
| | | between light source | between the light source and |
| | | and cantilever. | the cantilever, make sure to |
| | | | tighten the nut in place. |
| | | Loose connector in | Retighten the connector on |
| | | cantilever shaft. | the cantilever shaft. |
| | | Control circuit | Notify the manufacturer or |
| | | damage. | local agent. |
| | | The Network power | Check the power supply |
| | | supply voltage is too | voltage of the network. If the |
| | | low. | voltage is too low, stop using |
| | | | the device. |
| 3 | Phosphoric screen of | Internal electrical | Notify the manufacturer or |
| | the light source does | failure of light | local agent. |
| | not work. | source. | |
| 4 | All light sources go on | Control circuit | Notify the manufacturer or |
| | after startup without | failure. | local agent. |
| | switching on the light | | |
| | source. | | |
| 5 | The time setting is out | Control circuit | Notify the manufacturer or |
| | of control. | failure. | local agent. |
| 6 | Repeatedly remind the | Communication line | Notify the manufacturer or |
| | user of the following | failure. | local agent. |
| | words: “The type of | | |
| | irradiator is abnormal, | | |
| | please restart”. | | |
| 7 | Remind the user of | Irradiance collection | Notify the manufacturer or |
| | the following words: | probe failure. | local agent. |
| | Automatic calibration | | |
| | value deviation. | | |

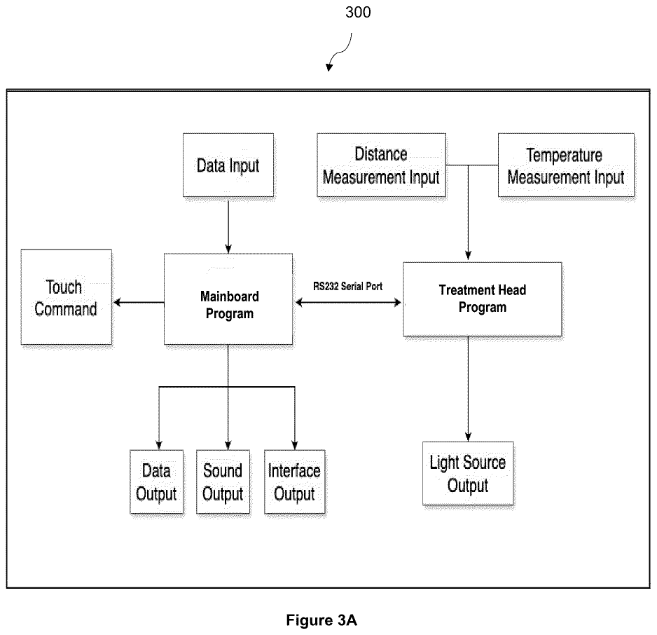

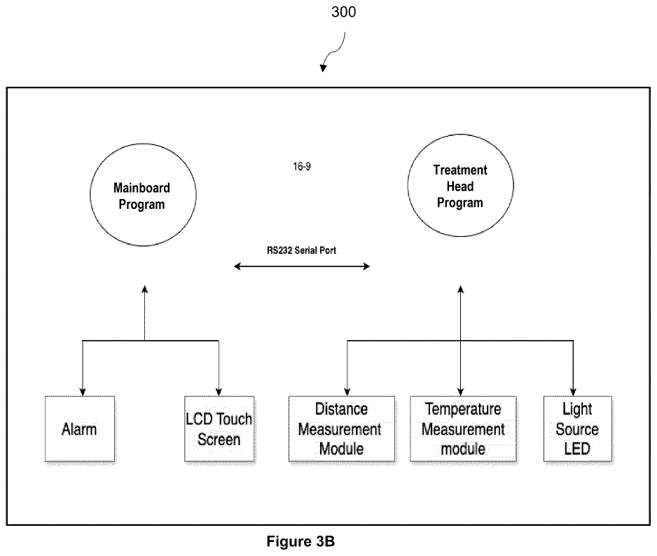

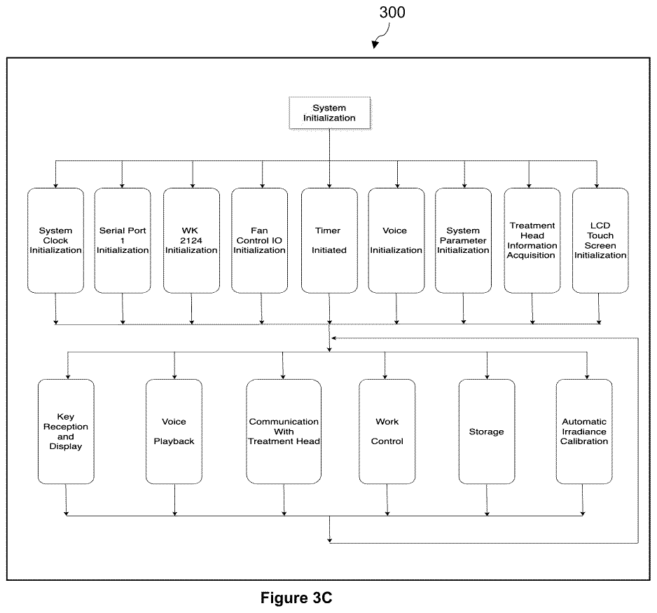

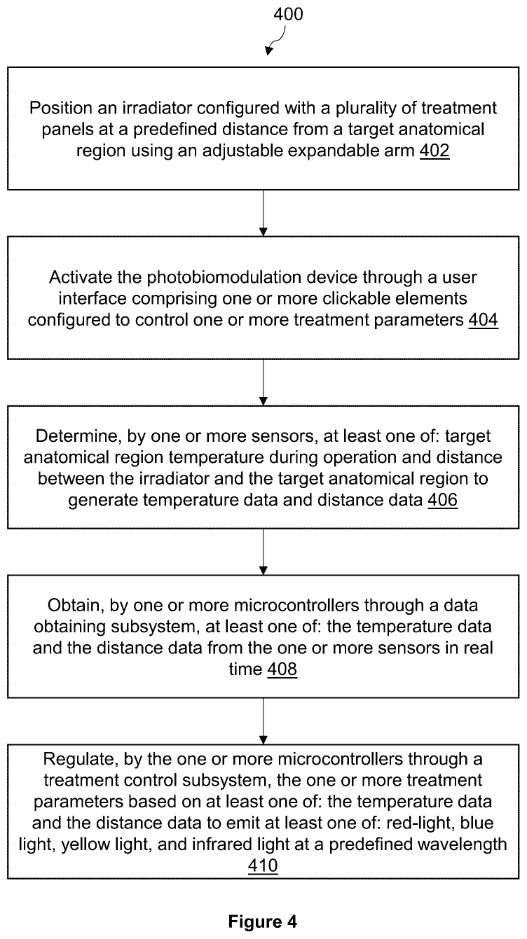

FIGS. 3A to 3C illustrate block diagrams 300 associated with the device 100 for accelerating the surgical rehabilitation, in accordance with an embodiment of the present disclosure.

In an exemplary embodiment, as shown in FIG. 3A, a mainboard program is a main control terminal, mainly responsible for receiving and processing at least one of: user commands, equipment information and data display, sound output, and interface output. A treatment head program is a processing end, mainly responsible for receiving the user commands and processing of at least one of: a device mainboard program, report equipment temperature measurement, distance measurement, treatment head parameters and other information.

In an exemplary embodiment, as shown in FIG. 3B, the mainboard program and the treatment head program are connected through a hardware RS232 serial port for data interaction. The mainboard program controls an alarm, the LCD touch screen 102, and other peripherals to realize the display of the interface 200. The treatment head program is connected with at least one of: a peripheral distance measurement module, a temperature measurement module, a light source LED module, and the like to realize the driving control of the irradiator 108.

In an exemplary embodiment, as shown in FIG. 3C, the computer-readable instructions associated with the mainboard program initialization is achieved by following steps: a) set a system clock frequency to 8 MHz for an external crystal oscillator and set the system clock to 72 MHZ after internal PLL frequency division, b) initialize usart1, working at 115200 bps, 8-bit data bit, 1-bit stop bit, no check. Communicate with the LCD screen 102, c) a WK2124 is initialized, and a serial port 1 is used to communicate with the treatment head program. The configuration is 9600 bps, 8-bit data bit, 1-bit stop bit, and no check, d) initialize and configure a fan control IO, e) the timer is initialized. TIM2 and TIM3 are mainly used to control the irradiator 108 by Pulse Width Modulation (PWM) output, and TIM4 is used to generate the basic timing of 10 ms, f) the initialization of peripheral circuits of AP89341K voice chip, g) read the system parameters to ensure that the device 100 operation parameters are correct, h) obtain the information of the treatment head to ensure the accuracy of the treatment output, and i) initialize the LCD screen 102 variable.

All the keys on the device 100 are in the form of a touch screen. When the key is pressed, the device 100 receives the touch screen key information in the way of interruption. When the read key is pressed, the value is converted into a predefined key value and reported. Display numbers and characters at specified positions according to the reading and writing sequence and command format of the LCD screen 102. The reading and writing timing of the LCD screen 102 is not complicated. The high and low pulse sequence of each signal on the LCD screen 102 interface is controlled according to the timing chart to simulate their timing relationship. As the LCD screen 102 is touch sensitive, the one or more surgeons only need to send an American Standard Code for Information Interchange (ASCII) code of the characters to be displayed to the user interface 120 data line.

A voice playback may control the opening and closing of voice through the setting interface. When the voice is on, the voice prompt may be given before starting the treatment and the music may be stopped at the end of the treatment. Communicate with the treatment unit to obtain the connection status of the treatment head, and at the same time, judge and update the working time of the treatment head and receive the information analysis of the treatment head. Start, pause, and stop of the irradiator 108 are controlled by pressing the key. When the treatment is finished, the record of the treatment is automatically saved, so that the patient may directly call it next time. The irradiance of the irradiator 108 may be one of: automatically calibrated and manually calibrated through the manufacturer's maintenance interface. If automatic calibration is selected, factory calibration may be conducted through the manufacturer's maintenance interface, and the irradiance calibration interface may be started. The calibration may also be started when the accumulated working time reaches 500 hours.

FIG. 4 illustrates a flow chart of a method 400 for accelerating the surgical rehabilitation using the device 100, in accordance with an embodiment of the present disclosure.

In another embodiment of the present disclosure, the method 400 for accelerating surgical rehabilitation using the device 100 is disclosed. At step 402, the method 400 includes positioning the irradiator 108 configured with the plurality of treatment panels 128 comprises the LEDs at the predefined distance from the target anatomical region using the adjustable expandable arm 122 connected to the mainframe 112 mounted on the mobile structure 110 with the plurality of lockable wheels 156. At step 404, the method 400 includes activating the device 100 through the user interface 120 comprising the one or more clickable elements configured to control the one or more treatment parameters comprising at least one of: the preset and named treatment designed protocols selection, the wavelength selection, the irradiance, the energy delivered in per unit area, the emission mode, the irradiator configuration mode, one of: the flat mode and the curved mode of the irradiator, and one of: the sequential mode and the concurrent mode, at the time of selecting the multiple wavelengths, and the treatment time.

At step 406, the method 400 includes determining, by the one or more sensors 134, at least one of: the target anatomical region temperature during operation and the distance between the irradiator 108 and the target anatomical region to generate the temperature data and the distance data. At step 408, the method 400 includes obtaining, by the one or more microcontrollers 136 through the data obtaining subsystem 146, at least one of: the temperature data and the distance data from the one or more sensors 134 in the real time. At step 410, the method 400 includes regulating, by the one or more microcontrollers 136 through the treatment control subsystem 154, the one or more treatment parameters based on at least one of: the temperature data and the distance data to emit at least one of: the red-light, the blue light, the yellow light, and the infrared light at the predefined wavelength.

In the next step, the method 400 include triggering, by the one or more microcontrollers 136 through the threshold-based safety interruption subsystem 148, at least one of: one or more alerts and one or more termination commands of the irradiator 108 if at least one of: the temperature data and the distance data falls outside the predefined threshold temperature data and the predefined operational distance range, respectively. In the next step, the method 400 include performing by the one or more microcontrollers 136 through the calibration subsystem 150, the comparative analysis between real-time predefined wavelength and factory-calibrated reference wavelength to detect output deviations in the predefined wavelength to calibrate each LED of the plurality of LEDs. In the next step, the method 400 includes logging, by the one or more microcontrollers 136 through the data logging subsystem 152, rehabilitation session data including at least one of: timestamp, selected wavelengths, energy delivered per unit area, irradiance, emission mode, and sensor readings into the one or more databases.

Numerous advantages of the present disclosure may be apparent from the discussion above. In accordance with the present disclosure, the device for accelerating the surgical rehabilitation is disclosed. The device is configured to revolutionize the field of plastic surgery by significantly improving pre-operative patient care and post-operative patient care. By employing specific wavelengths of light energy at controlled dosages, the device accelerates recovery, reduces the inflammation, swelling, and bruising, and enhances lymphatic drainage through targeted photobiomodulation. The device is exclusively tailored for surgical applications, providing one or more surgeons with an innovative tool to improve patient outcomes and minimize downtime. The blue light initiates the production of the ROS, which selectively targets bacterial cells due to the weaker antioxidant defenses, resulting in membrane disruption and bacterial death. The yellow light stimulates the mitochondria, resulting in the increased ATP, which fuels cellular repair and regeneration processes crucial for post-operative recovery. The light treatments effectively reduce the pro-inflammatory cytokines and enhance anti-inflammatory cytokine IL-10, which reduces the edema, accelerates recovery, and mitigates pain and swelling.

The device provides an innovative solution for surgical recovery by seamlessly integrating targeted wavelengths and dosages into pre-op, post-op, and maintenance protocols. This approach optimizes the healing environment, reduces the risks of infection, and improves overall patient outcomes in a minimally invasive manner. Clinical protocols and studies have demonstrated that targeted Photobiomodulation therapy significantly reduces recovery time in surgical patients compared to traditional care methods. The device provides flexibility with different pre-op, post-op, and maintenance protocols tailored to individual patient needs, enhancing the efficacy and personalization of care. By reducing bruise, swelling, and discomfort, the one or more patients experience faster healing with less visible side effects, leading to higher satisfaction rates. Unlike conventional treatments, which may involve medication and invasive methods, the device provides a pain-free, drug-free approach.

The device is employed for enhanced recovery in following procedures such as facial plastic, aesthetic and cosmetic surgeries (ex: Facelift, Neck-lift, Rhinoplasty and eyelid surgeries), body plastic surgeries (liposuction and tummy tuck), and breast augmentation. The device is employed as an adjunct therapy for reducing inflammation and pain post-operation, to assist in managing post-surgical edema and accelerating recovery.

This approach ensures the one or more patients feel well-cared for, allowing the one or more patients to participate in and observe the healing journey actively. This leads to improved outcomes and heightened patient satisfaction rates. The configuration of the device is tailored to meet the unique requirements of one or more surgeons. The device employs cutting-edge Surface Mount Device-Light Emitting Diode (SMD-LED) technology that requires zero maintenance and zero consumables for a lifetime of 50,000 hours. The device provides five customizable treatment modes, enabling the one or more surgeons to adjust parameters based on individual patient needs. The device provides a wide range of selectable wavelengths, allowing the one or more surgeons to target various skin concerns with accuracy and effectiveness. The device provides adjustable intensity levels, ensuring optimal treatment results while considering patient comfort and sensitivity. The device is ergonomically designed to provide ease of use and enhance the ability of the one or more surgeons to operate treatments efficiently.

When the voltage fluctuation of the power supply is too large, an AC voltage stabilizer with an accuracy of 2 percent is provided. The device provides customizable treatment modes, enabling the one or more surgeons to adjust parameters based on individual patient needs and specific skin conditions. Five programmable treatment settings allow saving customized times and doses, and stored treatments to avoid repeated settings. The selectable wavelengths allow the one or more surgeons to target various skin concerns with accuracy and effectiveness.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

What is claimed is:

1. A photobiomodulation device for accelerating surgical rehabilitation, comprising:
a mainframe operatively mounted on a mobile structure with a plurality of lockable wheels, configured to house at least one of: one or more electronic components and power sockets;
an irradiator configured with a plurality of treatment panels operatively connected to the mainframe on a first side through an adjustable expandable arm,
each treatment panel of the plurality of treatment panels comprises a plurality of light-emitting diodes (LEDs),
the plurality of LEDs configured to emit at least one of: red-light, blue light, yellow light, and infrared light,
each LED of the plurality of LEDs operate at a predefined wavelength; and
the adjustable expandable arm configured to provide at least one of: a vertical direction and a horizontal direction to the irradiator for reaching a target anatomical region;
one or more sensors operatively positioned on one or more treatment panels of the plurality of treatment panels, configured to determine at least one of: target anatomical region temperature during operation and distance between the irradiator and the target anatomical region for generating temperature data and distance data;
a user interface operatively mounted on the mainframe at a first end, configured to control one or more treatment parameters comprise at least one of: preset and named treatment designed protocols selection, wavelength selection, irradiance, energy delivered in per unit area, emission mode, irradiator configuration mode, one of: a flat mode and a curved mode of the irradiator, and one of: a sequential mode and a concurrent mode, at a time of selecting multiple wavelengths, and treatment time;
one or more microcontrollers;
a memory unit operatively connected to the one or more microcontrollers, wherein the memory unit comprises a set of computer-readable instructions in form of a plurality of subsystems, configured to be executed by the one or more microcontrollers, wherein the plurality of subsystems comprises:
a data obtaining subsystem configured to obtain at least one of: the temperature data and the distance data from the one or more sensors in real time;
a threshold-based safety interruption subsystem configured to trigger at least one of: one or more alerts and one or more termination commands of the irradiator if at least one of: the temperature data and the distance data falls outside a predefined threshold temperature data and a predefined operational distance range respectively;
a calibration subsystem configured to calibrate each LED of the plurality of LEDs by performing a comparative analysis between real-time predefined wavelength and factory-calibrated reference wavelength for detecting output deviations in the predefined wavelength;
a data logging subsystem configured to log rehabilitation session data including at least one of: timestamp, selected wavelengths, energy delivered per unit area, irradiance, emission mode, and sensor readings into one or more databases; and
a treatment control subsystem configured to regulate the one or more treatment parameters based on at least one of: the temperature data and the distance data for emitting at least one of: the red-light, the blue light, the yellow light, and the infrared light at the predefined wavelengths,
the emitted at least one of: the red-light, the blue light, the yellow light, and the infrared light, configured to stimulate at least one of: mitochondrial activity, mitigate bacterial loads, regulate sebaceous gland activity, modulate inflammatory cytokines to avert inflammation, promote lymphatic drainage, boost cellular growth and immune responses and enhance cellular regeneration at the target anatomical region for accelerating surgical rehabilitation.

2. The photobiomodulation device of claim 1, wherein
the plurality of LEDs comprises 280 surface-mounted LEDs for each treatment panel of the plurality of treatment panels, arranged in a predefined matrix pattern; and
the plurality of treatment panels comprise five treatment panels, each treatment panel of the plurality of treatment panels capable of being folded inward to conform to a curvature of one of: a human face and body contour.

3. The photobiomodulation device of claim 1, wherein the adjustable expandable arm configured to rotate positive 80 degrees and negative 80 degrees from a longitudinal axis of the mainframe; and
the adjustable expandable arm configured to tilt positive 15 degrees and negative 15 degrees from a pivotal point of the adjustable expandable arm;

4. The photobiomodulation device of claim 1, wherein
the predefined wavelengths of the red-light ranges between 623 nanometers (nm) and 643 nm;
the predefined wavelengths of the blue light ranges between 407 nm and 427 nm;
the predefined wavelengths of the yellow light ranges between 580 nm and 600 nm; and
the predefined wavelengths of the infrared light ranges between 825 nm and 845 nm.

5. The photobiomodulation device of claim 1, wherein at least one of: the red-light, the blue light, the yellow light, and the infrared light are emitted in a pulsed mode with defined frequencies of 2 Hertz (Hz), 5 Hz, and 10 Hz at flashing interval time of 0.5 seconds(s), 0.2 s and 0.1 s, respectively,
at least one of: the red-light, the blue light, the yellow light, and the infrared light are emitted in one of: a concurrent mode and a sequential mode, with multiple wavelength emission.

6. The photobiomodulation device of claim 1, wherein the one or more sensors comprises at least one of: a temperature sensor embedded at a second side of the central treatment panel of the plurality of treatment panels to determine the target anatomical region temperature, and a distance sensor integrated at a third side of the central treatment panel of the plurality of treatment panels to determine the distance between the irradiator and the target anatomical region.

7. The photobiomodulation device of claim 1, wherein the user interface configured to provide at least one of: one or more clickable elements, one or more voice comment inputs, one or more touchscreen inputs, one or more gesture-based controls, one or more remote-control inputs, and one or more programmable physical buttons, comprise at least one of:
a wavelength selector for choosing one or more wavelengths to be emitted by the irradiator;
a power level selector for regulating the irradiance delivered to the target anatomical region;
a treatment duration timer for setting the treatment time;
a treatment dosage input for setting a dose of the energy delivered in per unit area;
a mode selector for switching between pulsed mode and continuous mode;
a treatment controller for one of: initiating, terminating, and pausing treatment;
an irradiator mode selector for switching between one of: the flat mode and a curved mode of the irradiator;
a treatment mode selector for switching between one of: the sequential mode and the concurrent mode, at the time of selecting multiple wavelengths, and treatment time; and
a preset protocol selector for applying predefined treatment parameters based on one of: a treatment type and a body region.

8. The photobiomodulation device of claim 1, wherein the calibration subsystem is configured to trigger one or more calibration alerts after every 500 hours of cumulative operation.

9. The photobiomodulation device of claim 1, wherein the calibration subsystem is configured to perform one of: an automatic calibration and a manual calibration of the plurality of LEDs based on one of: predefined usage time and user initiation.

10. The photobiomodulation device of claim 1, wherein the treatment control subsystem configured to dynamically regulate treatment duration based on the real-time irradiance values and the selected energy delivered per unit area.

11. A method for accelerating surgical rehabilitation using a photobiomodulation device, comprising:
positioning an irradiator configured with a plurality of treatment panels comprise a plurality of light-emitting diodes (LEDs) at a predefined distance from a target anatomical region using an adjustable expandable arm connected to a mainframe mounted on a mobile structure with a plurality of lockable wheels;
activating the photobiomodulation device through a user interface comprising one or more clickable elements configured to control one or more treatment parameters comprising at least one of: preset and named treatment designed protocols selection, wavelength selection, irradiance, energy delivered in per unit area, emission mode, irradiator configuration mode, one of: a flat mode and a curved mode of the irradiator, and one of: a sequential mode and a concurrent mode, at a time of selecting multiple wavelengths, and treatment time;
determining, by one or more sensors, at least one of: target anatomical region temperature during operation and distance between the irradiator and the target anatomical region to generate temperature data and distance data;
obtaining, by one or more microcontrollers through a data obtaining subsystem, at least one of: the temperature data and the distance data from the one or more sensors in real time; and
regulating, by the one or more microcontrollers through a treatment control subsystem, the one or more treatment parameters based on at least one of: the temperature data and the distance data to emit at least one of: red-light, blue light, yellow light, and infrared light at a predefined wavelength,
wherein the emitted at least one of: the red-light, the blue light, the yellow light, and the infrared light, configured to stimulate at least one of: mitochondrial activity, mitigate bacterial loads, regulate sebaceous gland activity, modulate inflammatory cytokines to avert inflammation, promote lymphatic drainage, boost cellular growth and immune responses and enhance cellular regeneration at the target anatomical region for accelerating surgical rehabilitation.

12. The method of claim 11, further comprising:
triggering, by the one or more microcontrollers through a threshold-based safety interruption subsystem, at least one of: one or more alerts and one or more termination commands of the irradiator if at least one of: the temperature data and the distance data falls outside a predefined threshold temperature data and a predefined operational distance range respectively;
performing, by the one or more microcontrollers through a calibration subsystem, a comparative analysis between real-time predefined wavelength and factory-calibrated reference wavelength to detect output deviations in the predefined wavelength to calibrate each LED of the plurality of LEDs; and
logging, by the one or more microcontrollers through a data logging subsystem, rehabilitation session data including at least one of: timestamp, selected wavelengths, energy delivered per unit area, irradiance, emission mode, and sensor readings into one or more databases.

13. The method of claim 12, wherein the calibration subsystem further configured to:
trigger one or more calibration alerts after every 500 hours of cumulative operation; and
perform one of: an automatic calibration and a manual calibration of the plurality of LEDs based on one of: predefined usage time and user initiation.

14. The method of claim 11, wherein
rotating the adjustable expandable arm to positive 80 degrees and negative 80 degrees from a longitudinal axis of the mainframe; and
tilting the adjustable expandable arm to positive 15 degrees and negative 15 degrees from a pivotal point of the adjustable expandable arm.

15. The method of claim 11, wherein
the predefined wavelengths of the red-light ranges between 623 nanometers (nm) and 643 nm;
the predefined wavelengths of the blue light ranges between 407 nm and 427 nm;
the predefined wavelengths of the yellow light ranges between 580 nm and 600 nm; and
the predefined wavelengths of the infrared light ranges between 825 nm and 845 nm.

16. The method of claim 11, wherein emitting at least one of: the red-light, the blue light, the yellow light, and the infrared light in a pulsed mode with defined frequencies of 2 Hertz (Hz), 5 Hz, and 10 Hz at flashing interval time of 0.5 seconds(s), 0.2 s and 0.1 s, respectively,
emitting at least one of: the red-light, the blue light, the yellow light, and the infrared light in one of: a concurrent mode and a sequential mode, with multiple wavelength emission.

17. The method of claim 11, wherein the one or more sensors comprises at least one of: a temperature sensor embedded at a second side of the central treatment panel of the plurality of treatment panels to determine the target anatomical region temperature, and a distance sensor integrated at a third side of the central treatment panel of the plurality of treatment panels to determine the distance between the irradiator and the target anatomical region.

18. The method of claim 11, wherein providing, by the user interface, at least one of: one or more clickable elements, one or more voice comment inputs, one or more touchscreen inputs, one or more gesture-based controls, one or more remote-control inputs, and one or more programmable physical buttons, comprise at least one of:
a wavelength selector for choosing one or more wavelengths to be emitted by the irradiator;
a power level selector for regulating the irradiance delivered to the target anatomical region;
a treatment duration timer for setting the treatment time;
a treatment dosage input for setting a dose of the energy delivered in per unit area;
the mode selector for switching between pulsed mode and continuous mode;
a treatment controller for one of: initiating, terminating, and pausing treatment;
an irradiator mode selector for switching between one of: the flat mode and a curved mode of the irradiator;
a treatment mode selector for switching between one of: the sequential mode and the concurrent mode, at the time of selecting multiple wavelengths, and treatment time; and
a preset protocol selector for applying predefined treatment parameters based on one of: a treatment type and a body region.

19. The method of claim 11, wherein further comprises:
dynamically regulating, by the treatment control subsystem, treatment duration based on the real-time irradiance values and the selected energy delivered per unit area.

20. A non-transitory computer-readable storage medium storing computer-executable instructions that, when executed by one or more microcontrollers, cause the one or more microcontrollers to perform operations for accelerating surgical rehabilitation, the operations comprising:
obtaining at least one of: temperature data and distance data from one or more sensors associated with a photobiomodulation device in real time;
triggering at least one of: one or more alerts and one or more termination commands of an irradiator associated with the photobiomodulation device if at least one of: the temperature data and the distance data falls outside a predefined threshold temperature data and a predefined operational distance range respectively;
performing a comparative analysis between real-time predefined wavelength of each light-emitting diode (LED) of a plurality of LEDs and factory-calibrated reference wavelength for detecting output deviations in predefined wavelengths to calibrate each LED of the plurality of LEDs in each treatment panel of the plurality of treatment panels of the irradiator;
logging rehabilitation session data including at least one of: timestamp, selected wavelengths, energy delivered per unit area, irradiance, emission mode, and sensor readings into one or more databases; and
regulating one or more treatment parameters based on at least one of: the temperature data and the distance data for emitting at least one of: red-light, blue light, yellow light, and infrared light at the predefined wavelengths by the plurality of LEDs,
the emitted at least one of: the red-light, the blue light, the yellow light, and the infrared light, configured to stimulate at least one of: mitochondrial activity, mitigate bacterial loads, regulate sebaceous gland activity, modulate inflammatory cytokines to avert inflammation, promote lymphatic drainage, boost cellular growth and immune responses and enhance cellular regeneration at the target anatomical region for accelerating surgical rehabilitation.
